(19) United States
(12) Patent Application Publication
(10) Pub. No.: US 2010/0298340 A1
(43) Pub. Date: Nov. 25, 2010

(54) Thiazol-Guanidine Derivatives Useful As A (Beta)-Related Pathologies

(75) Inventors: Stefan Berg, Sodertalje (SE); Karin Kolmodin, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignees: AstraZeneca AB, Södertälje (SE); Astex Therapeutics Ltd., Cambridge (GB)

(21) Appl. No.: 12/296,771

(22) PCT Filed: Apr. 12, 2007
(86) PCT No.: PCT/SE2007/000347

§ 371 Date: Oct. 10, 2008

Related U.S. Application Data
(60) Provisional application No. 60/791,772, filed on Apr. 13, 2006.

(51) Int. Cl. A61K 31/497; C07C 335/28; A61K 31/175; C07D 277/56; A61K 31/426; C07D 277/48; C07D 417/12; A61K 31/433; A61K 31/454; C07D 417/10; C07D 417/14; A61K 31/4439; C07D 241/26; A61K 31/4965; A61P 25/00; A61P 25/28; C12N 9/99

(52) U.S. Cl. 514/254.02; 564/30; 514/581; 548/194; 514/370; 548/198; 548/181; 548/127; 514/361; 546/209; 514/326; 544/369; 546/270.7; 514/342; 544/407; 514/255.06; 544/405; 514/255.05; 435/184

(57) ABSTRACT

This invention relates to novel compounds having the structural formula I below: and to their pharmaceutically acceptable salt, compositions and methods of use. These novel compounds provide a treatment or prophylaxis of cognitive impairment, Alzheimer Disease, neurodegeneration and dementia.

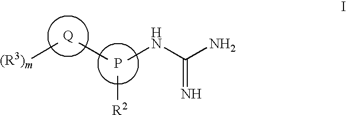

The present invention relates to novel compounds, their pharmaceutical compositions, methods of use and processes to make such compounds. In addition, the present invention relates to therapeutic methods for the treatment and/or prevention of Aβ-related pathologies such as in Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have (3-secretase activity (Hussain et al., 1999; Lin et. al, 2000; Yan et. al, 1999; Sinha et. al., 1999 and Vassar et. al., 1999). (3-secretase is also known in the literature as Asp2 (Yan et. al, 1999), Beta site APP Cleaving Enzyme (BACE)(Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041404, WO06/0065204, US2006287294, WO06/138265, WO06/138217, WO06/138230, WO06/138264, WO06/138266, WO06/099379, US20070004786, US20070004730, WO07/011,833, WO07/011,810).

### DISCLOSURE OF THE INVENTION

Provided herein are novel compounds of structural Formula I:

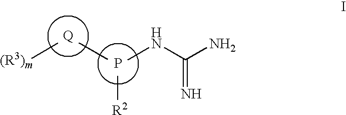

wherein

P is thiazole;

Q is independently selected from phenyl, thiazole, C₀₋₃alkylCONR⁴R⁵, C₀₋₃alkylNR⁴COR⁵, CO₀₋₃alkylNR⁴(SO₂)R⁵, and C₀₋₃alkyl(SO₂)NR⁴R⁵;

R² is independently selected from hydrogen, halogen, C₁₋₆alkyl, CN, C₀₋₆alkylOR⁴, fluoromethyl, difluoromethyl, trifluoromethyl, fluoromethoxy, difluoromethoxy, trifluoromethoxy, C₂₋₆alkenyl and C₂₋₆alkynyl;

R³ is independently selected from halogen, nitro, CHO, CN, OC₁₋₆alkylCN, OR⁴, OC₁₋₆alkylOR⁴, fluoromethyl, difluoromethyl, trifluoromethyl, fluoromethoxy, difluoromethoxy, trifluoromethoxy, NR⁴R⁵, OC₁₋₆alkylNR⁴R⁵, NR⁴COR⁵, CO₂R⁴, CONR⁴R⁵, OC₁₋₆alkylCONR⁴R⁵, OC₁₋₆alkylNR⁴(CO)R⁵, NR⁴(CO)R⁵, O(CO)NR⁴R⁵, NR⁴(CO)OR⁵, NR⁴(CO)NR⁴R⁵, O(CO)R⁴, COR⁴, OC₁₋₆alkylCOR⁴, SR⁴, (SO₂)NR⁴R⁵, OC₁₋₆alkylNR⁴(SO₂)R⁵, OC₀₋₆alkyl(SO₂)NR⁴R⁵, (SO)NR⁴R⁵, OC₁₋₆alkyl(SO)NR⁴R⁵, NR⁴(SO)R⁵, NR⁴(SO₂)R⁵, OC₁₋₆alkylNR⁴(SO)R⁵, OC₀₋₆alkylSO₂R⁴, SO₂R⁴, SOR⁴, C₁₋₆alkyl, C₂₋₆alkenyl, C₂₋₆alkynyl, C₃₋₆cycloalkyl, aryl and heteroaryl, wherein said C₁₋₆alkyl, C₂₋₆alkenyl, C₂₋₆alkynyl, C₃₋₆cycloalkyl, aryl or heteroaryl may be optionally substituted with one or more A;

R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylC₃₋₆heterocyclyl, C₀₋₆alkylaryl, C₀₋₆alkylheteroaryl and C₁₋₆alkylNR⁶R⁷, wherein said C₁₋₆alkyl, C₂₋₆alkenyl, C₀₋₆alkylC₃₋₆heterocyclyl, CO₀₋₆alkylaryl or C₀₋₆alkylheteroaryl may be optionally substituted by one or more A; or

R⁴ and R⁵ may together form a 5 or 6 membered heterocyclic ring containing one or more heteroatoms selected from N, O or S, which heterocyclic ring may be optionally substituted by one or more A;

A is independently selected from oxo, halogen, nitro, CN, OR⁶, C₁₋₆alkyl, C₀₋₆alkylaryl, heteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, NR⁶R⁷, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₀₋₆alkylaryl, heteroaryl, C₀₋₆alkylheterocyclyl or C₀₋₆alkylC₃₋₆cycloalkyl may be optionally substituted with one or more substituents independently selected from halogen, nitro, cyano, OR⁶, fluoromethyl, difluoromethyl, trifluoromethyl, fluoromethoxy, difluoromethoxy, trifluoromethoxy, and NR⁶R⁷;

R⁶ and R⁷ are independently selected from hydrogen, C₁₋₆alkyl, C₀₋₆alkylaryl, fluoromethyl, difluoromethyl and trifluoromethyl, or

R⁶ and R⁷ may together form an optionally substituted 5, 6 or 7 membered heterocyclic ring containing one or more heteroatoms selected from N, O or S;

m is 0, 1, 2 or 3;

provided that when Q is C₀₋₃alkylCONR⁴R⁵ and R⁴ or R⁵ is C₀₋₆alkylC₃₋₆heterocyclyl, said C₀₋₆alkylC₃₋₆heterocyclyl is not benzotriazole; and

provided that when Q is C₀₋₃alkylCONR⁴R⁵, C₀₋₃alkylNR⁴COR⁵, C₀₋₃alkylNR⁴(SO₂)R⁵ or C₀₋₃alkyl(SO₂)NR⁴R⁵, m is 0;

as a free base or a pharmaceutically acceptable salt, solvate or solvate of a salt thereof.

In one aspect of the invention, there is provided a compound of formula I:

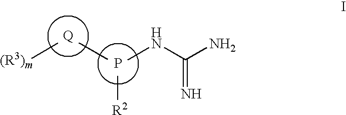

wherein

P is thiazole;

Q is independently selected from phenyl, thiazole, C₀₋₃alkylCONR⁴R⁵, C₀₋₃alkylNR⁴COR⁵, C₀₋₃alkylNR⁴(SO₂)R⁵, and C₀₋₃alkyl(SO₂)NR⁴R⁵;

R² is independently selected from hydrogen, halogen, C₁₋₆alkyl, CN, C₀₋₆alkylOR⁴, fluoromethyl, difluoromethyl, trifluoromethyl, fluoromethoxy, difluoromethoxy, trifluoromethoxy, C₂₋₆alkenyl and C₂₋₆alkynyl;

R³ is independently selected from halogen, nitro, CHO, CN, OC₁₋₆alkylCN, OR⁴, OC₁₋₆alkylOR⁴, fluoromethyl, difluoromethyl, trifluoromethyl, fluoromethoxy, difluoromethoxy, trifluoromethoxy, NR⁴R⁵, OC₁₋₆alkylNR⁴R⁵, NR⁴COR⁵, CO₂R⁴, CONR⁴R⁵, OC₁₋₆alkylCONR⁴R⁵, OC₁₋₆alkylNR⁴(CO)R⁵, NR⁴(CO)R⁵, O(CO)NR⁴R⁵, NR⁴(CO)OR⁵, NR⁴(CO)NR⁴R⁵, O(CO)R⁴, COR⁴, OC₁₋₆allylCOR⁴, SR⁴, (SO₂)NR⁴R⁵, OC₁₋₆alkylNR⁴(SO₂)R⁵, OC₀₋₆alkyl(SO₂)NR⁴R⁵, (SO)NR⁴R⁵, OC₁₋₆alkyl(SO)NR⁴R⁵, SO₃R⁴, NR⁴(SO)R⁵, NR⁴(SO₂)R⁵, OC₁₋₆allylNR⁴(SO)R⁵, OC₀₋₆alkylSO₂R⁴, SO₂R⁴, SOR⁴, C₁₋₆alkyl, C₂₋₆alkenyl, C₂₋₆alkynyl, C₃₋₆cycloalkyl, aryl and heteroaryl, wherein said C₁₋₆alkyl, C₂₋₆alkenyl, C₂₋₆alkynyl, C₃₋₆cycloalkyl, aryl or heteroaryl may be optionally substituted with one or more A;

R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylC₃₋₆heterocyclyl, C₀₋₆alkylaryl, C₀₋₆alkylheteroaryl and C₁₋₆alkylNR⁶R⁷, wherein said C₁₋₆alkyl, C₂₋₆alkenyl, C₀₋₆alkylC₃₋₆heterocyclyl, C₀₋₆alkylaryl or C₀₋₆alkylheteroaryl may be optionally substituted by one or more A; or R⁴ and R⁵ may together form a 5 or 6 membered heterocyclic ring containing one or more heteroatoms selected from N, O or S, which heterocyclic ring may be optionally substituted by one or more A;

A is independently selected from oxo, halogen, nitro, CN, OR⁶, C₁₋₆alkyl, C₀₋₆alkylaryl, heteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, NR⁶R⁷, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₅alkyl, C₀₋₆alkylaryl, heteroaryl, C₀₋₆alkylheterocyclyl or C₀₋₆alkylC₃₋₆cycloalkyl may be optionally substituted with one or more substituents independently selected from halogen, nitro, cyano, OR⁶, fluoromethyl, difluoromethyl, trifluoromethyl, fluoromethoxy, difluoromethoxy, trifluoromethoxy, and NR⁶R⁷;

R⁶ and R⁷ are independently selected from hydrogen, C₁₋₆alkyl, C₀₋₆alkylary fluoromethyl, difluoromethyl and trifluoromethyl, or

R⁶ and R⁷ may together form an optionally substituted 5, 6 or 7 membered heterocyclic ring containing one or more heteroatoms selected from N, O or S;

m is 0, 1, 2 or 3;

provided that when Q is C₀₋₃alicylCONR⁴R⁵ and R⁴ or R⁵ is C₀₋₆alkylC₃₋₆heterocyclyl, said C₀₋₆alkylC₃₋₆heterocyclyl is not benzotriazole; and

provided that when Q is C₀₋₃alkylCONR⁴R⁵, C₀₋₃alkyINR⁴COR⁵, C₀₋₃alkylNR⁴(SO₂)R⁵ or C₀₋₃alkyl(SO₂)NR⁴R⁵, in is 0; and

provided that the following compounds are excluded:
1-[2′-(Aminomethyl)-4,4′-bi-1,3-thiazol-2-yl]guanidine;
N-[(2′-{[Amino(imino)methyl]amino}-4,4′-bi-1,3-thiazol-2-yl)methyl]acetamide;
N-[(2-{[Amino(imino)methyl]amino}-4,5′-bi-1,3-thiazol-2′-yl)methyl]acetamide;
1-[2′-(Aminomethyl)-4,5′-bi-1,3-thiazol-2-yl]guanidine;
2′-{[Amino(imino)methyl]amino}-4,4′-bi-1,3-thiazole-2-carboxamide;
1-{2′-[(Dimethylamino)methyl]-4,4′-bi-1,3-thiazol-2-yl}guanidine;
1-(2′-Cyano-4,4′-bi-1,3-thiazol-2-yl)guanidine;
1-[2′-(Cyanomethyl)-4,4′-bi-1,3-thiazol-2-yl]guanidine;
N-(2′-{[Amino(imino)methyl]amino}-4,4′-bi-1,3-thiazol-2-yl)acetamide;
Ethyl 2′-{[amino(imino)methyl]amino}-4,4′-bi-1,3-thiazole-2-carboxylate;
1-[2′-(2-Aminoethyl)-4,4′-bi-1,3-thiazol-2-yl]guanidine;
N-[2-(2′-{[Amino(imino)methyl]amino}-4,4′-bi-1,3-thiazol-2-yl)ethyl]acetamide;
1-[4′-Methyl-2′-(methylamino)-4,5′-bi-1,3-thiazol-2-yl]guanidine;
1-{2′-[Formyl(methyl)amino]-4′-methyl-4,5′-bi-1,3-thiazol-2-yl}guanidine;
1-(2′-Amino-4,4′-bi-1,3-thiazol-2-yl)guanidine;
1-(4,4′-Bi-1,3-thiazol-2-yl)guanidine;
1-[2′-(Methylamino)-4,4′-bi-1,3-thiazol-2-yl]guanidine;
1-(4-Phenyl-1,3-thiazol-2-yl)guanidine;
1-[4-(3-Aminophenyl)-1,3-thiazol-2-yl]guanidine;
N-[3-(2-{[Amino(imino)methyl]amino}-1,3-thiazol-4-yl)phenyl]acetamide;
1-{4-[3-(2-Oxopropyl)phenyl]-1,3-thiazol-2-yl}guanidine;
N-[3-(2-{[Amino(imino)methyl]amino}-1,3-thiazol-5-yl)phenyl]acetamide;
1-[4-(4-tert-Butylphenyl)-1,3-thiazol-2-yl]guanidine;
1-[4-(4-Nitrophenyl)-1,3-thiazol-2-yl]guanidine;
1-[4-(4-Fluorophenyl)-1,3-thiazol-2-yl]guanidine;
1-[4-(4-Hydroxyphenyl)-1,3-thiazol-2-yl]guanidine;
1-[4-(4-Aminophenyl)-1,3-thiazol-2-yl]guanidine;
1-[4-(4-Methoxyphenyl)-1,3-thiazol-2-yl]guanidine;
1-[4-(2-Chlorophenyl)-1,3-thiazol-2-yl]guanidine;
1-[4-(3-Chlorophenyl)-1,3-thiazol-2-yl]guanidine;
1-[4-(2-Methylphenyl)-1,3-thiazol-2-yl]guanidine;
1-[4-(3-Methylphenyl)-1,3-thiazol-2-yl]guanidine;
1-(4-{3-[(Dimethylamino)methyl]phenyl}-1,3-thiazol-2-yl)guanidine;
1-[4-(2-Methoxyphenyl)-1,3-thiazol-2-yl]guanidine;
1-[4-(3-Hydroxyphenyl)-1,3-thiazol-2-yl]guanidine;
1-[4-(3,4-Dihydroxyphenyl)-1,3-thiazol-2-yl]guanidine;
1-[4-(3,4-Dihydroxyphenyl)-5-methyl-1,3-thiazol-2-yl]guanidine;
1-[4-(4-Isopropylphenyl)-1,3-thiazol-2-yl]guanidine;
1-[4-(3-Methoxyphenyl)-1,3-thiazol-2-yl]guanidine;
N-[3-(2-{[Amino(imino)methyl]amino}-1,3-thiazol-4-yl)benzyl]acetamide;
N-(3-{[4-(2-{[Amino(imino)methyl]amino}-1,3-thiazol-4-yl)phenyl]amino}-3-oxopropyl)-N-benzylbenzamide;
1-[4-(4-Chlorophenyl)-1,3-thiazol-2-yl]guanidine;
1-[4-(4-Methylphenyl)-1,3-thiazol-2-yl]guanidine;
1-{4-[3-(Dimethylamino)phenyl]-1,3-thiazol-2-yl}guanidine;
1-(4-{2-[(Dimethylanaino)methyl]phenyl}-1,3-thiazol-2-yl)guanidine;
1-(4-Biphenyl-4-yl-1,3-thiazol-2-yl)guanidine;
1-{4-[4-(Dimethylamino)phenyl]-1,3-thiazol-2-yl}guanidine;
1-[4-(3,4-Dimethoxyphenyl)-1,3-thiazol-2-yl]guanidine;
1-[4-(2-Hydroxyphenyl)-1,3-thiazol-2-yl]guanidine;
6-{[3-(2-{[Amino(imino)methyl]amino}-4-methyl-1,3-thiazol-5-yl)phenyl]amino}-6-oxohexanoic acid;
6-{[4-(2-{[Amino(imino)methyl]amino}-4-methyl-1,3-thiazol-5-yl)phenyl]amino}-6-oxohexanoic acid;
4-{[4-(2-{[Amino(imino)methyl]amino}-4-methyl-1,3-thiazol-5-yl)benzoyl]amino}-3-(4-chlorophenyl)butanoic acid;
4-{[3-(2-{[Amino(imino)methyl]amino}-4-methyl-1,3-thiazol-5-yl)benzoyl]amino}-3-(4-chlorophenyl)butanoic acid;
3-{[4-(2-{[Amino(imino)methyl]amino}-4-methyl-1,3-thiazol-5-yl)benzoyl]amino}-3-phenylpropanoic acid;
3-{[3-(2-{[Amino(imino)methyl]amino}-4-methyl-1,3-thiazol-5-yl)benzoyl]amino}-3-phenylpropanoic acid;
4-{[3-(2-{[Amino(imino)methyl]amino}-1,3-thiazol-4-yl)benzoyl]amino}-3-(4-chlorophenyl)butanoic acid;
5-{[3-(2-{[Amino(imino)methyl]amino}-4-methyl-1,3-thiazol-5-yl)phenyl]amino}-3-(4-methoxyphenyl)-5-oxopentanoic acid;
5-{[3-(2-{[Amino(imino)methyl]amino}-4-methyl-1,3-thiazol-5-yl)phenyl]amino}-5-oxo-3-pyridin-3-ylpentanoic acid;
5-{[3-(2-{[Amino(imino)methyl]amino}-4-ethyl-1,3-thiazol-5-yl)phenyl]amino}-5-oxo-3-phenylpentanoic acid;
5-{[3-(2-{[Amino(imino)methyl]amino}-4-propyl-1,3-thiazol-5-yl)phenyl]amino}-5-oxo-3-phenylpentanoic acid;
5-{[3-(2-{[Amino(imino)methyl]amino}-4-butyl-1,3-thiazol-5-yl)phenyl]amino}-5-oxo-3-phenylpentanoic acid;
5-{[3-(2-{[Amino(imino)methyl]amino}-4-pentyl-1,3-thiazol-5-yl)phenyl]amino}-5-oxo-3-phenylpentanoic acid;
5-{[3-(2-{[Amino(imino)methyl]amino}-4-methyl-1,3-thiazol-5-yl)phenyl]amino}-3-(4-chlorophenyl)-5-oxopentanoic acid;
5-{[3-(2-{[Amino(imino)methyl]amino}-4-methyl-1,3-thiazol-5-yl)phenyl]amino}-3-hydroxy-5-oxopentanoic acid;
5-{[3-(2-{[Amino(imino)methyl]amino}-4-methyl-1,3-thiazol-5-yl)phenyl]amino}-5-oxopentanoic acid;
5-{[3-(2-{[Amino(imino)methyl]amino}-4-methyl-1,3-thiazol-5-yl)phenyl]amino}-3-methyl-5-oxopentanoic acid;
5-{[3-(2-{[Amino (imino)methyl]amino}-4-methyl-1,3-thiazol-5-yl)phenyl]amino}-3-(3-bromophenyl)-5-oxopentanoic acid:
5-{[3-(2-{[Amino (imino)methyl]amino}-4-methyl-1,3-thiazol-5-yl)phenyl]amino}-3-(3,5-dichloro-2-hydroxyphenyl)-5-oxopentanoic acid;
5-{[3-(2-{[Amino (imino)methyl]amino}-4-methyl-1,3-thiazol-5-yl)phenyl]amino}-3-(3-methoxyphenyl)-5-oxopentanoic acid;
5-{[3-(2-{[Amino (imino)methyl]amino}-4-methyl-1,3-thiazol-5-yl)-4-chlorophenyl]amino}-5-oxo-3-phenylpentanoic acid;
5-{[5-(2-{[Amino(imino)methyl]amino}-4-methyl-1,3-thiazol-5-yl)-2-chlorophenyl]amino}-5-oxo-3-phenylpentanoic acid;
4-{[3-(2-{[Amino(imino)methyl]amino}-4-methyl-1,3-thiazol-5-yl)benzoyl]amino}-2-[(butylsulfonyl)amino]butanoic acid;
3-({[3-(2-{[Amino(imino)methyl]amino}-4-methyl-1,3-thiazol-5-yl)phenyl]carbamoyl}amino)-3-phenylpropanoic acid;
5-{[3-(2-{[Amino(imino)methyl]amino}-4-methyl-1,3-thiazol-5-yl)phenyl](methyl)amino}-5-oxo-3-phenylpentanoic acid;
4-{[4-(2-{[Amino(imino)methyl]amino}-4-methyl-1,3-thiazol-5-yl)phenyl]amino}-4-oxobutanoic acid;
4-{[3-(2-{[Amino(imino)methyl]amino}-4-methyl-1,3-thiazol-5-yl)phenyl]amino}-4-oxobutanoic acid;
5-{[3-(2-{[Amino(imino)methyl]amino}-4-methyl-1,3-thiazol-5-yl)phenyl]amino}-5-oxo-3-phenylpentanoic acid;
5-{[4-(2-{[Amino(imino)methyl]amino}-4-methyl-1,3-thiazol-5-yl)phenyl]amino}-5-oxo-3-phenylpentanoic acid;
1-[4-Methyl-5-(4-nitrophenyl)-1,3-thiazol-2-yl]guanidine;
1-[4-Methyl-5-(3-nitrophenyl)-1,3-thiazol-2-yl]guanidine;
Methyl 4-(2-{[amino(imino)methyl]amino}-4-methyl-1,3-thiazol-5-yl)benzoate;
Methyl 3-(2-{[amino(imino)methyl]amino}-4-methyl-1,3-thiazol-5-yl)benzoate;
1-[5-(3-Aminophenyl)-4-methyl-1,3-thiazol-2-yl]guanidine;
1-[5-(4-Aminophenyl)-4-methyl-1,3-thiazol-2-yl]guanidine;
4-(2-{[Amino(imino)methyl]amino}-4-methyl-1,3-thiazol-5-yl)benzoic acid;
3-(2-{[Amino(imino)methyl]amino}-4-methyl-1,3-thiazol-5-yl)benzoic acid;
Methyl 6-{[4-(2-{[amino(imino)methyl]amino}-4-methyl-1,3-thiazol-5-yl)phenyl]amino}-6-oxohexanoate;
Methyl 6-{[3-(2-{[amino(imino)methyl]amino}-4-methyl-1,3-thiazol-5-yl)phenyl]amino}-6-oxohexanoate;
Ethyl 4-{[4-(2-{[amino(imino)methyl]amino}-4-methyl-1,3-thiazol-5-yl)benzoyl]amino}-3-(4-chlorophenyl)butanoate;
Ethyl 4-{[3-(2-{[amino(imino)methyl]amino}-4-methyl-1,3-thiazol-5-yl)benzoyl]amino}-3-(4-chlorophenyl)butanoate;
Ethyl 3-{[4-(2-{[amino(imino)methyl]amino}-4-methyl-1,3-thiazol-5-yl)benzoyl]amino}-3-phenylpropanoate;
Ethyl 3-{[3-(2-{[amino(imino)methyl]amino}-4-methyl-1,3-thiazol-5-yl)benzoyl]amino}-3-phenylpropanoate;
5-{[3-(2-{[Amino(imino)methyl]amino}-1,3-thiazol-4-yl)phenyl]amino}-5-oxo-3-phenylpentanoic acid;
1-[4-(3-Nitrophenyl)-1,3-thiazol-2-yl]guanidine;
3-(2-{[Amino(imino)methyl]amino}-1,3-thiazol-4-yl)benzoic acid;
1-[4-Ethyl-5-(3-nitrophenyl)-1,3-thiazol-2-yl]guanidine;
1-[5-(3-Aminophenyl)-4-ethyl-1,3-thiazol-2-yl]guanidine;
1-[5-(3-Aminophenyl)-4-propyl-1,3-thiazol-2-yl]guanidine;
1-[5-(3-Nitrophenyl)-4-propyl-1,3-thiazol-2-yl]guanidine;
1-[4-Butyl-5-(3-nitrophenyl)-1,3-thiazol-2-yl]guanidine;
1-[5-(3-Aminophenyl)-4-butyl-1,3-thiazol-2-yl]guanidine;
1-[5-(3-Aminophenyl)-4-pentyl-1,3-thiazol-2-yl]guanidine;
1-[5-(3-Nitrophenyl)-4-pentyl-1,3-thiazol-2-yl]guanidine;
1-[5-(2-Chloro-5-nitrophenyl)-4-methyl-1,3-thiazol-2-yl]guanidine;
1-[5-(4-Chloro-3-nitrophenyl)-4-methyl-1,3-thiazol-2-yl]guanidine;
Ethyl 3-({[3-(2-{[amino(imino)methyl]amino}-4-methyl-1,3-thiazol-5-yl)phenyl]carbamoyl}amino)-3-phenylpropanoate;
Ethyl 5-{[3-(2-{[amino(imino)methyl]amino}-4-methyl-1,3-thiazol-5-yl)phenyl]amino}-5-oxo-3-phenylpentanoate;
1-[5-(3-{[(2,5-Dioxopyrrolidin-1-yl)methyl]amino}phenyl)-4-methyl-1,3-thiazol-2-yl]guanidine;
1-{4-Methyl-5-[3-(methylamino)phenyl]-1,3-thiazol-2-yl}guanidine;
1-[4-(3-{4-[Methyl(phenyl)amino]butoxy}phenyl)-1,3-thiazol-2-yl]guanidine;
1-{4-[3-(4-Chlorobutoxy)phenyl]-1,3-thiazol-2-yl}guanidine;
1-[4-(3-{4-[(4-Bromophenyl)(methyl)amino]butoxy}phenyl)-1,3-thiazol-2-yl]guanidine;
1-{4-[2-(4-Chloro-2-methylphenoxy)phenyl]-1,3-thiazol-2-yl}guanidine;
1-{4-[2-(2,4-Dimethylphenoxy)phenyl]-1,3-thiazol-2-yl}guanidine;
1-{4-[2-(4-Cyclohexylphenoxy)phenyl]-1,3-thiazol-2-yl}guanidine;
1-[4-(3,5-di-tert-Butyl-4-hydroxyphenyl)-1,3-thiazol-2-yl]guanidine;
1-(4-Biphenyl-3-yl-1,3-thiazol-2-yl)guanidine;
1-[4-(4-Phenoxyphenyl)-1,3-thiazol-2-yl]guanidine;
1-[4-(4-Benzylphenyl)-1,3-thiazol-2-yl]guanidine;
1-[4-(4-Cyclohexylphenyl)-1,3-thiazol-2-yl]guanidine;
1-[4-(3-Chloro-2-hydroxy-4,6-dimethoxyphenyl)-1,3-thiazol-2-yl]guanidine;
1-[4-(2-Naphthyl)-1,3-thiazol-2-yl]guanidine;
1-(4-Biphenyl-2-yl-1,3-thiazol-2-yl)guanidine;
4-(2-{[Amino(imino)methyl]amino}-5-methyl-1,3-thiazol-4-yl)phenyl pivalate;
1-[4-(4-Hydroxyphenyl)-5-methyl-1,3-thiazol-2-yl]guanidine;
1-(5-Methyl-4-phenyl-1,3-thiazol-2-yl)guanidine;
1-(5-Ethyl-4-phenyl-1,3-thiazol-2-yl)guanidine;
1-{4-[3-(Trifluoromethyl)phenyl]-1,3-thiazol-2-yl}guanidine;
1-[4-(2,3,4-Trihydroxyphenyl)-1,3-thiazol-2-yl]guanidine;
1-[4-(2,5-Dihydroxyphenyl)-1,3-thiazol-2-yl]guanidine;
4-(2-{[Amino(imino)methyl]amino}-1,3-thiazol-4-yl)-1,2-phenylene diacetate;
N-[4-(2-{[Amino(imino)methyl]amino}-1,3-thiazol-4-yl)benzyl]acetamide;
N-[4-(2-{[Amino(imino)methyl]amino}-1,3-thiazol-4-yl)-2-methylbenzyl]acetamide;
N-[4-(2-{[Amino(imino)methyl]amino}-1,3-thiazol-4-yl)-2-chlorobenzyl]acetamide;
N-[5-(2-{[Amino(imino)methyl]amino}-1,3-thiazol-4-yl)-2-methoxybenzyl]acetamide;
1-{4-[3-(Aminomethyl)-4-methoxyphenyl]-1,3-thiazol-2-yl}guanidine;
1-[4-(3-Cyanophenyl)-1,3-thiazol-2-yl]guanidine;
1-(4-{3-[5-(Trifluoromethyl)-1,2,4-oxadiazol-3-yl]phenyl}-1,3-thiazol-2-yl)guanidine;
1-{4-[3-(Methylamino)phenyl]-1,3-thiazol-2-yl}guanidine;
1-(4-{3-[(3-Amino-1,2,4-oxadiazol-5-yl)amino]phenyl}-1,3-thiazol-2-yl)guanidine;
1-[4-(3-{[3-(Methylamino)-1,2,4-oxadiazol-5-yl]amino}phenyl)-1,3-thiazol-2-yl]guanidine;
1-(4-{4-[1-(2-Morpholin-4-ylethyl)-1H-benzimidazol-2-yl]phenyl}-1,3-thiazol-2-yl)guanidine;
1-(4-{4-[1-(1-Ethylpiperidin-3-yl)-1H-benzimidazol-2-yl]phenyl}-1,3-thiazol-2-yl)guanidine;
1-(4-{4-[1-(3-Morpholin-4-ylpropyl)-1H-benzimidazol-2-yl]phenyl}-1,3-thiazol-2-yl)guanidine;
1-[5-Methyl-4-(3-nitrophenyl)-1,3-thiazol-2-yl]guanidine;
1-[4-(2-Aminophenyl)-1,3-thiazol-2-yl]guanidine;
1-[4-(4-Cyanophenyl)-1,3-thiazol-2-yl]guanidine;
1-{4-[2-(2-pyrrolidin-1-ylethoxy)phenyl]-1,3-thiazol-2-yl}guanidine;
1-{5-Methyl-4-[4-(2-pyrrolidin-1-ylethoxy)phenyl]-1,3-thiazol-2-yl}guanidine;
1-{4-[3-Amino-4-(benzyloxy)phenyl]-1,3-thiazol-2-yl}guanidine;
1-(4-{3-[2-(Dimethylamino)ethoxy]phenyl}-1,3-thiazol-2-yl)guanidine;
1-{5-Methyl-4-[2-(2-pyrrolidin-1-ylethoxy)phenyl]-1,3-thiazol-2-yl}guanidine;
1-{4-[3-(3-Cyanopropoxy)phenyl]-1,3-thiazol-2-yl}guanidine;
1-[5-Ethyl-4-(3-nitrophenyl)-1,3-thiazol-2-yl]guanidine;
3-(2-{[Amino(imino)methyl]amino}-1,3-thiazol-4-yl)benzenesulfonamide;
1-[4-(3,4,5-Trimethoxyphenyl)-1,3-thiazol-2-yl]guanidine;
1-[4-(3-Hydroxyphenyl)-5-methyl-1,3-thiazol-2-yl]guanidine;
3-(2-{[Amino(imino)methyl]amino}-5-methyl-1,3-thiazol-4-yl)phenyl acetate;
1-{4-[4-(Benzyloxy)-3-nitrophenyl]-1,3-thiazol-2-yl}guanidine;
1-[4-(2-Nitrophenyl)-1,3-thiazol-2-yl]guanidine;
1-[4-(2-Hydroxyphenyl)-5-methyl-1,3-thiazol-2-yl]guanidine;
4-(2-{[Amino(imino)methyl]amino}-1,3-thiazol-4-yl)phenyl acetate;
Methyl 5-(2-{[amino(imino)methyl]amino}-1,3-thiazol-4-yl)-2-hydroxybenzoate;
1-[4-(3-Aminophenyl)-5-methyl-1,3-thiazol-2-yl]guanidine;
1-[4-(3-Aminophenyl)-5-ethyl-1,3-thiazol-2-yl]guanidine;
1-(4-{3-[3-(Dimethylamino)propoxy]phenyl}-1,3-thiazol-2-yl)guanidine;
1-{4-[4-(2-Pyrrolidin-1-ylethoxy)phenyl]-1,3-thiazol-2-yl}guanidine;
5-(2-{[Amino(imino)methyl]amino}-1,3-thiazol-4-yl)-2-hydroxy-N-methylbenzamide;
3-(2-{[Amino (imino)methyl]amino}-1,3-thiazol-4-yl)benzamide;
1-{4-[4-(Cyanomethyl)phenyl]-1,3-thiazol-2-yl}guanidine;
1-(4-{4-[3-(Trifluoromethyl)-1,2,4-oxadiazol-5-yl]phenyl}-1,3-thiazol-2-yl)guanidine;
Methyl 4-(2-{[amino(imino)methyl]amino}-1,3-thiazol-4-yl)benzoate;
1-[4-(4-Methyl-3-nitrophenyl)-1,3-thiazol-2-yl]guanidine;
1-[4-(4-Chloro-3-nitrophenyl)-1,3-thiazol-2-yl]guanidine;
1-[4-(4-Methoxy-3-nitrophenyl)-1,3-thiazol-2-yl]guanidine;
1-[4-(3-Amino-4-methoxyphenyl)-1,3-thiazol-2-yl]guanidine;
1-[4-(3-Amino-4-chlorophenyl)-1,3-thiazol-2-yl]guanidine;
1-[4-(3-Amino-4-methylphenyl)-1,3-thiazol-2-yl]guanidine;
3-(2-{[Amino(imino)methyl]amino}-1,3-thiazol-4-yl)-N-methylbenzamide;
1-{4-[3-(1H-Imidazol-2-ylamino)phenyl]-1,3-thiazol-2-yl}guanidine;
1-(4-{3-[(Methylamino)methyl]phenyl}-1,3-thiazol-2-yl)guanidine;
1-[4-(3-Amino-4-fluorophenyl)-1,3-thiazol-2-yl]guanidine;
1-[4-(2-Amino-5-bromophenyl)-1,3-thiazol-2-yl]guanidine;
1-[4-(2-Methyl-5-nitrophenyl)-1,3-thiazol-2-yl]guanidine;
1-[4-(3-Fluoro-4-nitrophenyl)-1,3-thiazol-2-yl]guanidine;
1-[4-(2-Bromo-5-nitrophenyl)-1,3-thiazol-2-yl]guanidine;
N-[3-(2-{[Amino(imino)methyl]amino}-1,3-thiazol-4-yl)phenyl]-2,2,2-trifluoroacetamide;
1-[4-(3-Formamidophenyl)-1,3-thiazol-2-yl]guanidine;
1-{4-[3-(4,5-Dihydro-1H-imidazol-2-ylamino)phenyl]-1,3-thiazol-2-yl}guanidine;
1-[4-(2-Amino-5-methylphenyl)-1,3-thiazol-2-yl]guanidine;
1-[4-(4-Bromophenyl)-1,3-thiazol-2-yl]guanidine;
1-[4-(4-Bromophenyl)-5-methyl-1,3-thiazol-2-yl]guanidine;
1-{4-[4-(Trifluoromethyl)phenyl]-1,3-thiazol-2-yl}guanidine;
1-[4-(2-Methyl-1H-indol-5-yl)-1,3-thiazol-2-yl]guanidine;
1-[4-(4-Chlorophenyl)-5-methyl-1,3-thiazol-2-yl]guanidine;
1-[4-(3-Aminophenyl)-5-butyl-1,3-thiazol-2-yl]guanidine;
1-[4-(3-Aminophenyl)-5-pentyl-1,3-thiazol-2-yl]guanidine;
4-(2-{[Amino(imino)methyl]amino}-5-methyl-1,3-thiazol-4-yl)phenyl acetate;
1-[4-(2,4,5-Trimethylphenyl)-1,3-thiazol-2-yl]guanidine;
1-{4-[3-(Cyanoamino)phenyl]-1,3-thiazol-2-yl}guanidine;
1-[4-(2-Oxo-2,3-dihydro-1H-indol-5-yl)-1,3-thiazol-2-yl]guanidine;
1-[5-Methyl-4-(2-oxo-2,3-dihydro-1H-indol-5-yl)-1,3-thiazol-2-yl]guanidine;
1-[4-(2,5-Dichlorophenyl)-1,3-thiazol-2-yl]guanidine;
1-[4-(4-Acetyl-6-chloro-3,4-dihydro-2H-1,4-benzoxazin-8-yl)-1,3-thiazol-2-yl]guanidine;
1-{4-[6-Chloro-3-oxo-4-(3-phenoxypropyl)-3,4-dihydro-2H-1,4-benzoxazin-8-yl]-1,3-thiazol-2-yl}guanidine;
1-[4-(1-Acetyl-2,3-dihydro-1H-indol-5-yl)-1,3-thiazol-2-yl]guanidine;
1-{4-[6-Chloro-4-(2-morpholin-4-ylethyl)-3-oxo-3,4-dihydro-2H-1,4-benzoxazin-8-yl]-1,3-thiazol-2-yl}guanidine;
1-[4-(2,4-Dichlorophenyl)-1,3-thiazol-2-yl]guanidine;
1-{4-[3-(1,3-Dioxo-1,3-dihydro-2H-isoindol-2-yl)phenyl]-1,3-thiazol-2-yl}guanidine;
1-[4-(1-Acetyl-2-methyl-2,3-dihydro-1H-indol-5-yl)-1,3-thiazol-2-yl]guanidine;
1-[4-(1,3-Benzodioxol-5-yl)-1,3-thiazol-2-yl]guanidine;
1-[5-Methyl-4-(1-propionyl-2,3-dihydro-1H-indol-5-yl)-1,3-thiazol-2-yl]guanidine;
1-[4-(2,4-Dimethylphenyl)-1,3-thiazol-2-yl]guanidine;
1-[4-(1-Isobutyryl-2,3-dihydro-1H-indol-5-yl)-1,3-thiazol-2-yl]guanidine;
1-[4-(1-Isobutyryl-2,3-dihydro-1H-indol-5-yl)-5-methyl-1,3-thiazol-2-yl]guanidine;
1-(4-{3-[(1,3-Dioxo-1,3-dihydro-2H-isoindol-2-yl)methyl]phenyl}-1,3-thiazol-2-yl)guanidine;
1-{4-[1-(Cyclopropylcarbonyl)-2,3-dihydro-1H-indol-5-yl]-1,3-thiazol-2-yl}guanidine;
1-{4-[1-(Cyclopropylcarbonyl)-2-methyl-2,3-dihydro-1H-indol-5-yl]-1,3-thiazol-2-yl}guanidine;
1-[4-(6-Chloro-4-methyl-3-oxo-3,4-dihydro-2H-1,4-benzoxazin-8-yl)-1,3-thiazol-2-yl]guanidine;
1-{4-[1-(Cyclohexylcarbonyl)-2,3-dihydro-1H-indol-5-yl]-5-methyl-1,3-thiazol-2-yl}guanidine;
1-{4-[1-(Methylsulfonyl)-2,3-dihydro-1H-indol-5-yl]-1,3-thiazol-2-yl}guanidine;
1-{-4-[2-Methyl-1-(methylsulfonyl)-2,3-dihydro-1H-indol-5-yl]-1,3-thiazol-2-yl}guanidine;
1-[4-(1-Acetyl-2,3-dihydro-1H-indol-5-yl)-5-methyl-1,3-thiazol-2-yl]guanidine;
1-{5-Methyl-4-[1-(methylsulfonyl)-2,3-dihydro-1H-indol-5-yl]-1,3-thiazol-2-yl}guanidine;
1-[4-(1-Propionyl-2,3-dihydro-1H-indol-5-yl)-1,3-thiazol-2-yl]guanidine;
1-[4-(2-Methyl-1-propionyl-2,3-dihydro-1H-indol-5-yl)-1,3-thiazol-2-yl]guanidine; 1-[4-(3-oxo-3,4-dihydro-2H-1,4-benzoxazin-6-yl)-1,3-thiazol-2-yl]guanidine;
1-[4-(2-Methyl-1H-imidazol-4-yl)-1,3-thiazol-2-yl]guanidine;
N-{[5-(2-{[Amino(imino)methyl]amino}-1,3-thiazol-4-yl)-2-furyl]methyl}acetamide;
1-(4-{5-[(Cyanoamino)methyl]-2-furyl}-1,3-thiazol-2-yl)guanidine;
N-{[5-(2-{[Amino(imino)methyl]amino}-1,3-thiazol-4-yl)-2-furyl]methyl}propanamide;
N-{[5-(2-{[Amino(imino)methyl]amino}-1,3-thiazol-4-yl)-2-furyl]methyl}butanamide;
N-{[5-(2-{[Amino(imino)methyl]amino}-1,3-thiazol-4-yl)-2-furyl]methyl}-2-methylpropanamide;
N-{[5-(2-{[Amino(imino)methyl]amino}-1,3-thiazol-4-yl)-2-furyl]methyl}pentanamide;
1-{4-[5-(Aminomethyl)-2-furyl]-1,3-thiazol-2-yl}guanidine;
N-{[5-(2-{[Amino(imino)methyl]amino}-1,3-thiazol-4-yl)-2-thienyl]methyl}acetamide;
N-[(2′-{[Amino(imino)methyl]amino}-4,4′-bi-1,3-thiazol-2-yl)methyl]acetamide;
N-[(2-{[Amino(imino)methyl]amino}-4,5′-bi-1,3-thiazol-2′-yl)methyl]acetamide;
N-{[5-(2-{[Amino(imino)methyl]amino}-1,3-thiazol-4-yl)-1,3,4-oxadiazol-2-yl]methyl}acetamide;
N-{[5-(2-{[Amino(imino)methyl]amino}-1,3-thiazol-4-yl)-4H-1,2,4-triazol-3-yl]methyl}acetamide;
1-{4-[5-(2-Methyl-1H-imidazol-5-yl)-2-furyl]-1,3-thiazol-2-yl}guanidine;
1-{4-[5-(5-Amino-4H-1,2,4-triazol-3-yl)-2-furyl]-1,3-thiazol-2-yl}guanidine;
Methyl 5-(2-{[amino(imino)methyl]amino}-1,3-thiazol-4-yl)-2-furoate;
Methyl[5-(2-{[amino(imino)methyl]amino}-1,3-thiazol-4-yl)-2-furyl]acetate;
1-(4-{5-[(1,3-Dioxo-1,3-dihydro-2H-isoindol-2-yl)methyl]-4H-1,2,4-triazol-3-yl}-1,3-thiazol-2-yl)guanidine;
1-{4-[(2,5-Dioxopyrrolidin-1-yl)carbonyl]-1,3-thiazol-2-yl}guanidine;
3-{[(2-{[Amino(imino)methyl]amino}-4-methyl-1,3-thiazol-5-yl)carbonyl]amino}benzoic acid;
Ethyl 3-{[(2-{[amino(imino)methyl]amino}-4-methyl-1,3-thiazol-5-yl)carbonyl]amino}benzoate;

as a free base or a pharmaceutically acceptable salt, solvate or solvate of a salt thereof.

In another aspect, the present invention provides pharmaceutical compositions comprising as active ingredient a therapeutically effective amount of a compound of formula I in association with pharmaceutically acceptable excipients, carriers or diluents.

In another aspect, the present invention provides a compound described herein or a pharmaceutically acceptable salt thereof, for use as a medicament.

In yet another aspect, the present invention provides a compound described herein or a pharmaceutically acceptable salt thereof, for use in treating or preventing an Aβ-related pathology.

In yet another aspect, the present invention provides a compound described herein or a pharmaceutically acceptable salt thereof, for use in treating or preventing an Aβ-related pathology wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In yet another aspect, the present invention provides a compound described herein in the manufacture of a medicament for treating or preventing an Aβ-related pathology.

In yet another aspect, the present invention provides a compound described herein in the manufacture of a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In yet another aspect, the present invention provides methods of inhibiting activity of BACE comprising contacting said BACE with a compound of Formula I.

In yet another aspect, the present invention provides methods of treating or preventing an Aβ-related pathology in a mammal, comprising administering to the patient a therapeutically effective amount of a compound of Formula I.

Said methods can also be methods of treating or preventing an Aβ-related pathology in a mammal, comprising administering to the patient a therapeutically effective amount of a compound of formula I and at least one cognitive enhancing agent, memory enhancing agent, anti-inflammatory agent or choline esterase inhibitor.

Said methods can also be methods of treating or preventing an Aβ-related pathology in a mammal, comprising administering to the patient a therapeutically effective amount of a compound of formula I in combination with an atypical antipsychotic agent.

Said methods refer to a mammal and said mammal may be a human.

Said Aβ-related pathology may be selected from Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy and cortical basal degeneration.

Cognitive enhancing agents, memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa)

Atypical antipsychotic agents includes, but not limited to, olanzapine (marketed as Zyprexa), aripiprazole (marketed as Abilify), risperidone (marketed as Risperdal), quetiapine (marketed as Seroquel), clozapine (marketed as Clozaril), ziprasidone (marketed as Geodon) and olanzapine/fluoxetine (marketed as Symbyax).

In another aspect of the invention, the compounds of the present invention are represented is by a method for the prophylaxis of Aβ-related pathologies comprising administering to a human a therapeutically effective amount of a compound of formula I or a pharmaceutically acceptable salt, thereof as defined herein.

In another aspect of the invention, the present invention provides that the mammal or human being treated with a compound of the invention has been diagnosed with a particular disease or disorder, such as those described herein. In these cases, the mammal or human being treated is in need of such treatment. Diagnosis, however, need not be previously performed.

In yet another aspect of the invention, there is provided a compound of formula I, wherein R² is selected from hydrogen and C₁₋₆alkyl.

In yet another aspect of the invention, there is provided a compound of formula I, wherein R² is selected from hydrogen, methyl and ethyl.

In yet another aspect of the invention, there is provided a compound of formula I, wherein wherein Q is selected from phenyl, thiazole and C₀₋₃alkylCONR⁴R⁵.

In yet another aspect of the invention, there is provided a compound of formula I, wherein wherein Q is thiazole.

In one embodiment of this aspect, there is provided a compound of formula I, wherein R² is selected from hydrogen and methyl.

In another embodiment of this aspect, there is provided a compound of formula I, wherein m is 1 or 2.

In yet another embodiment of this aspect, there is provided a compound of formula I, wherein R³ is independently selected from NR⁴R⁵, C₁₋₆allyl and heteroaryl, wherein said heteroaryl may be optionally substituted with one or more A.

In yet another embodiment of this aspect, there is provided a compound of formula I, wherein R³ is NR⁴R⁵, wherein said R⁴ and R⁵ may represent hydrogen.

In yet another embodiment of this aspect, there is provided a compound of formula I, wherein R³ is heteroaryl, wherein said heteroaryl may be substituted with one or more A. An example is represented by R³ being pyridine, substituted with two halogens. Alternatively, said A may be independently selected from NR⁶R⁷ and hydrogen and said R⁶ and R⁷ may represent hydrogen.

In yet another embodiment of this aspect, there is provided a compound of formula I, wherein R³ is NR⁴R⁵, wherein said R⁴ may represent hydrogen and said R⁵ may represent aryl, said aryl optionally being substituted with one or more A, said A being selected from halogen, nitro, CN, OR⁶, NR⁶R⁷, COR⁶ and CO₂R⁶. R⁶ and R⁷ may independently be selected from hydrogen and methyl. R⁶ may be represented by C₀₋₆alkylaryl.

In yet another embodiment of this aspect, there is provided a compound of formula I, wherein R³ is NR⁴R⁵, wherein said R⁴ and R⁵ may represent methyl.

In yet another embodiment of this aspect, there is provided a compound of formula I, wherein R³ is NR⁴R⁵, wherein said R⁴ and R⁵ may independently be selected from hydrogen and C₂₋₆alkenyl.

In yet another embodiment of this aspect, there is provided a compound of formula I, wherein R³ is NR⁴R⁵, wherein said R⁴ and R⁵ may independently be selected from hydrogen and C₀₋₆alkylheteroaryl, such as pyridine.

In yet another aspect of the invention, there is provided a compound of formula I, wherein wherein Q is phenyl.

In one embodiment of this aspect, there is provided a compound of formula I, wherein R² is selected from hydrogen and methyl.

In another embodiment of this aspect, there is provided a compound of formula I, wherein m is 1.

In yet another embodiment of this aspect, there is provided a compound of formula I, wherein R³ is selected from nitro, NR⁴R⁵, NR⁴COR⁵, CONR⁴R⁵ and NR⁴(SO₂)R⁵.

In yet another embodiment of this aspect, there is provided a compound of formula I, wherein R³ is NR⁴R⁵, wherein said R⁴ and R⁵ are independently selected from C₀₋₆alkylaryl and C₀₋₆alkylC₃₋₆heterocyclyl.

In yet another embodiment of this aspect, there is provided a compound of formula I, wherein R³ is NR⁴R⁵, wherein said R⁴ and R⁵ are independently selected from hydrogen and C₀₋₆alkylaryl, said C₀₋₆alkylaryl optionally substituted with one or more A.

Said A may independently be selected from OR⁶, CO₂R⁶ and halogen, wherein said R⁶ may be selected from hydrogen and C₀₋₆alkylaryl.

In yet another embodiment of this aspect, there is provided a compound of formula I, wherein R³ is NR⁴R⁵, wherein said R⁴ and R⁵ together form a 5 membered heterocyclic ring containing one N heteroatom.

In yet another embodiment of this aspect, there is provided a compound of formula I, wherein R³ is NR⁴R⁵, wherein said R⁴ represents hydrogen and said R⁵ represents C₂₋₆alkenyl optionally substituted with one A. Said A may represent C₀₋₆alkylaryl, substituted with NR⁶NR⁷, said R⁶ and R⁷ being C₁₋₆alkyl.

In yet another embodiment of this aspect, there is provided a compound of formula I, wherein R³ is NR⁴(SO₂)R⁵, wherein said R⁴ may represent hydrogen and said R⁵ may represent C₀₋₆alkylaryl, said C₀₋₆alkylaryl being substituted with one or more A. Said A may represent C₀₋₆alkylaryl, substituted with one OR⁶ wherein R⁶ represents methyl.

In yet another embodiment of this aspect, there is provided a compound of formula I, wherein R³ is NR⁴COR⁵, wherein R⁴ may represent hydrogen and said R⁵ may represent C₀₋₆alkylaryl or C₀₋₆alkylheteroaryl, said C₀₋₆alkylaryl or C₀₋₆alkylheteroaryl being substituted with one or more A. Said A may independently be selected from halogen, OR⁶, C₁₋₆alkyl, C₀₋₆alkylaryl, heteroaryl, said C₁₋₆alkyl, C₀₋₆alkylaryl and heteroaryl being optionally substituted with NR⁶R⁷, wherein said R⁶ and R⁷ may independently be selected from methyl and C₀₋₆alkylaryl.

In yet another embodiment of this aspect, there is provided a compound of formula I, wherein R³ is R³ is CONR⁴R⁵ wherein said R⁴ and R⁵ may independently be selected from hydrogen and C₀₋₆alkylC₃₋₆heterocyclyl, alternatively, said R⁴ and R⁵ may together form a 6 membered heterocyclic ring containing one or more N heteroatoms, which heterocyclic ring is substituted by one or more A. Said A may be C₀₋₆alkylaryl optionally substituted with OR⁶ wherein R⁶ may represent hydrogen.

In yet another aspect of the invention, there is provided a compound of formula I, wherein wherein Q is C₀₋₃alkylCONR⁴R⁵, and m is 0. Said R⁴ may be hydrogen and said R⁵ may be CO₀₋₆ alkylaryl. Alternatively, said R⁴ may be hydrogen and said R⁵ may be C₀₋₆alkylheteroaryl, optionally substituted with C₁₋₆alkyl.

In yet another aspect of the invention, there is provided a compound selected from:
2-{[Amino(imino)methyl]amino}-N-1-anthryl-1,3-thiazole-4-carboxamide;
2-{[Amino(imino)methyl]amino}-N-(9-ethyl-9H-carbazol-3-yl)-1,3-thiazole-4-carboxamide;
N-[3-(2-{[Amino(imino)methyl]amino}-4-methyl-1,3-thiazol-5-yl)phenyl]-4-(benzyloxy)benzamide;
N-[3-(2-{[Amino(imino)methyl]amino}-4-methyl-1,3-thiazol-5-yl)phenyl]-4′-methoxybiphenyl-4-sulfonamide;
N-[3-(2-{[Amino(imino)methyl]amino}-4-methyl-1,3-thiazol-5-yl)phenyl]-4-(1,2,3-thiadiazol-4-yl)benzamide;
N-[3-(2-{[Amino(imino)methyl]amino}-4-methyl-1,3-thiazol-5-yl)phenyl]-3-chloro-6-ethyl-1-benzothiophene-2-carb oxamide;
N-[3-(2-{[Amino(imino)methyl]amino}-4-methyl-1,3-thiazol-5-yl)phenyl]-2-naphthamide;
Benzyl 4-({[4-(2-{[amino(imino)methyl]amino}-4-ethyl-1,3-thiazol-5-yl)phenyl]amino}methyl)piperidine-1-carboxylate;
N″-{5-[4-(Dibenzylamino)phenyl]-4-ethyl-1,3-thiazol-2-yl}guanidine;
N-(5-{3-[(2-Bromo-6-hydroxybenzyl)amino]phenyl}-4-methyl-1,3-thiazol-2-yl)guanidine;
N-[5-(4-{[4-(2-Hydroxyphenyl)piperazin-1-yl]carbonyl}phenyl)-4-methyl-1,3-thiazol-2-yl]guanidine;
N-{2′-[(3,4-Dichlorophenyl)amino]-4-methyl-5,5′-bi-1,3-thiazol-2-yl}guanidine;
N-{4-Methyl-2′-[(4-nitrophenyl)amino]-5,5′-bi-1,3-thiazol-2-yl}guanidine;
N-{2′-[(4-Cyanophenyl)amino]-4-methyl-5,5′-bi-1,3-thiazol-2-yl}guanidine;
N-{2-[(4-Hydroxyphenyl)amino]-4-methyl-5,5′-bi-1,3-thiazol-2-yl}guanidine;
4-[(2′-{[Amino(imino)methyl]amino}-4′-methyl-5,5′-bi-1,3-thiazol-2-yl)amino]benzoic acid;
N-{2′-[(4-Acetylphenyl)amino]-4-methyl-5,5′-bi-1,3-thiazol-2-yl}guanidine;
N-{2′-[(2,4-Dimethoxyphenyl)amino]-4-methyl-5,5′-bi-1,3-thiazol-2-yl}guanidine;
4-(2-{[Amino(imino)methyl]amino}-4-methyl-1,3-thiazol-5-yl)-N-(4-pyrrolidin-1-ylbutyl)benzamide;
Benzyl 4-{[(4-{2-[(diaminomethylene)amino]-4-methyl-1,3-thiazol-5-yl}phenyl)amino]methyl}piperidine-1-carboxylate;
2-(5-{4-[Benzyl(piperidin-4-ylmethyl)amino]phenyl}-4-methyl-1,3-thiazol-2-yl)guanidine;
2-{5-[4-({(2E)-3-[4-(Dimethylamino)phenyl]prop-2-en-1-yl}amino)phenyl]-4-methyl-1,3-thiazol-2-yl}guanidine;
1-[5-(2-Nitrophenyl)-1,3-thiazol-2-yl]guanidine;
1-[4-(4-Pyrrolidin-1-ylphenyl)-1,3-thiazol-2-yl]guanidine;
1-{2-[(4-Aminophenyl)amino]-4′-methyl-4,5′-bi-1,3-thiazol-2′-yl}guanidine;
1-{4′-Methyl-2-[(4-phenoxyphenyl)amino]-4,5′-bi-1,3-thiazol-2′-yl}guanidine;
1-{2-[(4-Methoxyphenyl)amino]-4′-methyl-4,5′-bi-1,3-thiazol-2′-yl}guanidine;
1-{4′-Methyl-2-[(4-nitrophenyl)amino]-4,5′-bi-1,3-thiazol-2′-yl}guanidine;
1-[4′-Methyl-2-(pyridin-4-ylamino)-4,5′-bi-1,3-thiazol-2′-yl]guanidine;
1-[2-(Dimethylamino)-4′-methyl-4,5′-bi-1,3-thiazol-2′-yl]guanidine;
1-[2-(Allylamino)-4′-methyl-4,5′-bi-1,3-thiazol-2′-yl]guanidine;
1-[2-(2,6-Dichloropyridin-4-yl)-4′-methyl-4,5′-bi-1,3-thiazol-2′-yl]guanidine;
N-[4-(2-{[Amino(imino)methyl]amino}-4-methyl-1,3-thiazol-5-yl)phenyl]-5-fluoro-1H-indole-2-carboxamide;
N-[4-(2-{[Amino(imino)methyl]amino}-4-methyl-1,3-thiazol-5-yl)phenyl]-4-{[methyl(phenyl)amino]methyl}benzamide;
2-{[Amino(imino)methyl]amino}-N-(9-oxo-9H-fluoren-2-yl)-1,3-thiazole-4-carboxamide;
2-{[Amino (imino)methyl]amino}-N-1H-indol-5-yl-1,3-thiazole-4-carboxamide;
1-(2′-Amino-4′-methyl-4,5′-bi-1,3-thiazol-2-yl)guanidine;
1-[2-(3,5-Diamino-6-chloropyrazin-2-yl)-4′-methyl-4,5′-bi-1,3-thiazol-2′-yl]guanidine;

as a free base or a pharmaceutically acceptable salt, solvate or solvate of a salt thereof.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of Formula I.

It is to be understood that the present invention relates to any and all tautomeric forms of the compounds of Formula I.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

The present invention further includes isotopically labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, 76Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

If the compound is anionic, or has a functional group that may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group —C(O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(O)OCH₃, —C(O)OCH₂CH₃, —C(O)OC(CH₃)₃, and —C(O)OPh. Examples of acyloxy (reverse ester) groups are represented by —OC(O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(O)CH₃ (acetoxy), —OC(O)CH₂CH₃, —OC(O)C(CH₃)₃, —OC(O)Ph, and —OC(O)CH₂Ph.

Derivatives that are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(O)OR) is cleaved to yield the active drug. Such esters may be formed by esterification, for example, of any of the carboxylic acid groups (—C(O)OH) in the parent compound, with, where appropriate, prior protection of any other reactive groups present in the parent compound, followed by deprotection if required.

Examples of such metabolically labile esters include those of the formula —C(O)OR wherein R is: C₁₋₇alkyl (e.g., -Me, -Et, -nPr, -iPr, -nBu, -sBu, -iBu, -tBu); C₁₋₇-aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound that, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Compounds of the present invention have been shown to inhibit beta secretase (including BACE) activity in vitro. Inhibitors of beta secretase have been shown to be useful in blocking formation or aggregation of Aβ peptide and therefore have a beneficial effect in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia. Hence compounds of the present invention and their salts are expected to be active against age-related diseases such as Alzheimer, as well as other Aβ related pathologies such as Downs syndrome and β-amyloid angiopathy. It is expected that the compounds of the present invention would most likely be used as a single agent but could also be used as in combination with a broad range of cognition deficit enhancement agents.

### DEFINITIONS

The definitions set forth in this application are intended to clarify terms used throughout this application. The term “herein” means the entire application.

As used in this application, the term “optionally substituted”, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halogen, CN, NH₂, OH, COOH, OC₁₋₆alkyl, CH₂OH, SO₂H, C₁₋₆alkyl, OC₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O) aryl, C(O)Oaryl, C(O)NHaryl, C(O)Naryl₂, SO₂aryl, SO₂NHaryl, SO₂Naryl₂, NHaryl, Naryl₂, NC(O)aryl, NC(O)aryl₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 6 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl. As used herein, “alkynyl” used alone or as a suffix or prefix is intended to include both branched and straight-chain alkyne containing aliphatic hydrocarbon groups having from 2 to 6 carbon atoms or if a specified number of carbon atoms is provided then that specific number would be intended. For example “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. For example 9-fluorenone.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 4 to 12 carbons atoms.

As used herein, “cycloalkynyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl. It is understood that when the total number of S and O atoms in the heterocyclyl exceeds 1, then these heteroatoms are not adjacent to one another. Ring structures containing 3 to 10 atoms would be a mono-, bi- or tricyclic heterocyclyls. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydropyranyl and 2,5-dioxoimidazolidinyl.

As used herein, “heteroaryl” or “heteroaromatic” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3r^{d} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric and the like; and the salts prepared from organic acids. The pharmaceutically acceptable salts of the present invention can be synthesized from the parent compound that contains a basic or acidic moiety by conventional chemical methods. Generally (but not necessarily), such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

As used herein, “in vivo hydrolysable precursors” means an in vivo hydrolysable (or cleavable) ester of a compound of Formula I that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism is where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Aβ-related pathologies include, but are not limited to: Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia.

Neurodegenerative Disorder(s) includes, but is not limited to, Alzheimer's Disease, Mild Cognitive Impairment, Dementia, Age-Associated Memory Impairment, Age-Related Cognitive Decline, Disorder(s) associated with neurofibrillar tangle pathologies, Dementia due to Alzheimer's Disease, Dementia due to Schizophrenia, Dementia due to Parkinson's Disease, Dementia due to Creutzfeld-Jacob Disease, Dementia due to Huntington's Disease, Dementia due to Pick's Disease, Stroke, Head Trauma, Spinal Injury, Multiple Sclerosis, Migraine, Pain, Systemic Pain, Localized Pain, Nociceptive Pain, Neuropathic Pain, Urinary Incontinence, Sexual Dysfunction, Premature Ejaculation, Motor Disorder(s), Endocrine Disorder(s), Gastrointestinal Disorder(s), and Vasospasm.

Many of the above conditions and disorder(s) are defined for example in the American Psychiatric Association Diagnostic and Statistical Manual of Mental Disorders, Fourth Edition, Text Revision, Washington, D.C., American Psychiatric Association, 2000.

### Pharmaceutical Compositions

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substance, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula I or a pharmaceutically acceptable salt thereof for the therapeutic treatment (including prophylactic treatment) of mammals including humans; it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The quantity of the compound to be administered will vary for the patient being treated and will vary from about 100 ng/kg of body weight to 100 mg/kg of body weight per day and preferably will be from 10 ng/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

### Methods of Preparation

The present invention also relates to processes for preparing the compound of formula I as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is understood that microwaves can be used for the heating of reaction mixtures.

### Preparation of Intermediates

The process, wherein P, Q, R², R³, R⁴, R⁵, R⁶ and R⁷, unless otherwise specified, are as hereinbefore defined, comprises,

(i) reaction of an α-halocarbonyl compound of formula II, wherein halo represents halogen e.g. iodine, bromine or chlorine, R⁸ is a group such as a ketone or an optionally substituted aryl and R⁹ is hydrogen or alkyl, with a compound of formula III to obtain a compound of formula IV, wherein R² is as defined above.

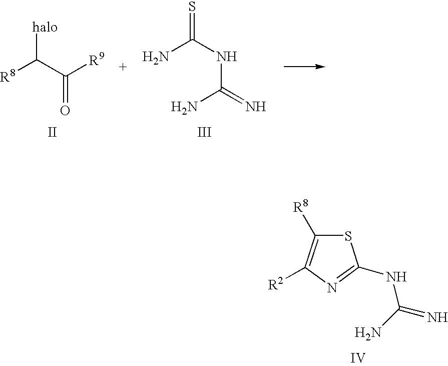

The reaction may be carried out by reaction of a compound of formula III, such as 1-[amino(imino)methyl]thiourea with a suitable α-halocarbonyl compound of formula II, such as ethyl bromopyruvate or bromo(2-nitrophenyl)acetaldehyde, in a suitable solvent such as methanol, ethanol, isopropanol, acetone, acetonitrile or N,N-dimethylformamide at a temperature between −78° C. and reflux.

(ii) reaction of an α-halocarbonyl compound of formula V, wherein halo represents halogen e.g. iodine, bromine or chlorine and R¹⁰ a group such as an optionally substituted aryl or an ester, with a compound of formula VI to obtain a compound of formula VII,

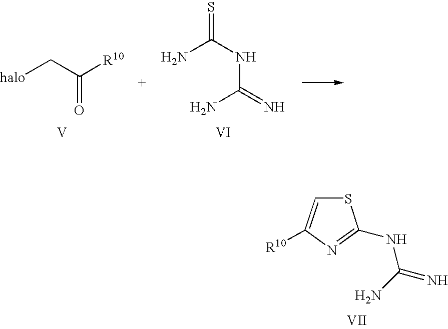

The reaction may be carried by reaction of a compound of formula VI such as 1-[amino(imino)methyl]thiourea with a suitable α-halocarbonyl compound of formula V, such as 3-bromopentane-2,4-dione or 2-bromo-1-(4-pyrrolidin-1-ylphenyl)ethanone, in a suitable solvent such as methanol, ethanol, isopropanol, acetone, acetonitrile or N,N-dimethylformamide at a temperature between −78° C. and reflux.

bromination of a compound of formula VIII, wherein R² is as defined above and R¹¹ is alkyl, to form a compound of formula IX.

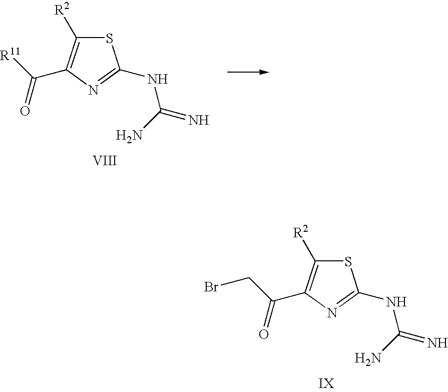

The reaction may be carried out by reaction with a suitable brominating agent such as bromine in a suitable solvent such as acetic acid, carbon tetrachloride or water at a temperature between 0° C. and reflux.

(iv) hydrolysis of a compound of formula VIII, wherein R² is as defined above and R¹¹ is alkoxy, to form a compound of formula X.

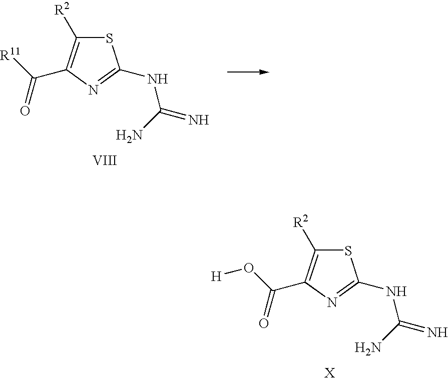

The reaction may be carried out with either base or acid in a suitable solvent such as water, tetrahydrofuran, ethanol or methanol at a temperature range between −20° C. and reflux. A suitable base may be an alkaline earth metal hydroxide such as lithium hydroxide, potassium hydroxide or sodium hydroxide. A suitable acid may be trifluoroacetic acid.

(v) conversion of a compound of formula XI, wherein Q is an optionally substituted aryl, to form a compound of formula XII, wherein R² is as defined above.

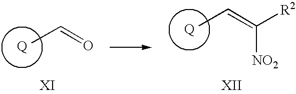

The reaction may be carried out by treating a compound of formula XI with an appropriate nitroalkane such as nitroethane or nitropropane and ammonium acetate in a suitable solvent such as acetic acid at a temperature between 0° C. and reflux.

(vi) conversion of a compound of formula XII, wherein Q is an optionally substituted aryl and R² is as defined above, to form a compound of formula XIII.

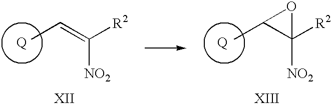

The reaction may be carried out by treating a compound of formula XII with an appropriate epoxidation agent such as an alkaline solution of hydrogen peroxide or t-butyl hydroperoxide or by an percarboxylic acid such as rn-chloroperbenzoic acid. The reaction may be carried out in a suitable solvent such as methanol, ethanol, tetrahydrofuran, dichloromethane or acetonitrile at a temperature between −78° C. and reflux.

(vii) reaction between a compound of formula III, and a compound of formula XIII, wherein Q is an optionally substituted aryl and R² is as defined above, to a compound of formula XIV.

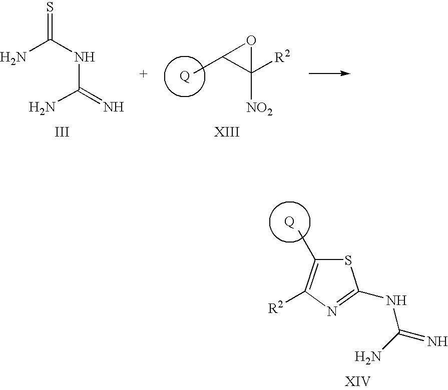

The reaction may be carried out by reaction of a suitable nitroepoxide (XIII) with a compound III such as 1-[amino(imino)methyl]thiourea in a suitable solvent such as ethanol, methanol, tetrahydrofuran, acetone or acetonitrile at a temperature between −20° C. and reflux.

(viii) reduction of a compound of formula XV, wherein R² is as defined above to a compound of formula XVI.

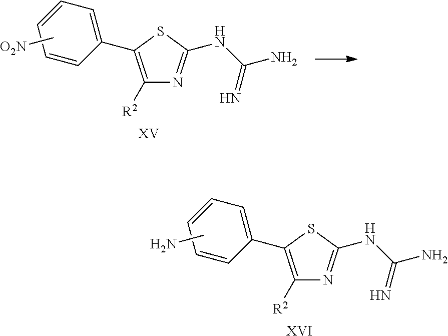

The reduction may be carried out using a suitable reducing agent such as palladium on carbon and ammonium formate or catalytic hydrogenation in an appropriate solvent such as methanol or ethanol. The reaction can be carried out at a temperature between 0° C. and reflux or in a microwave oven at a temperature between room temperature and 200° C.

(ix) hydrolysis of a compound of formula XVII, wherein R² is as defined above, to a compound of formula XVIII.

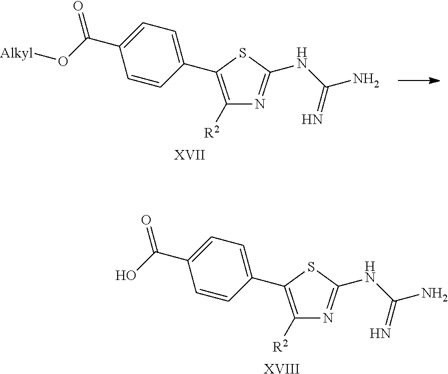

The reaction may be carried out with either base or acid in a suitable solvent such as water, tetrahydrofuran, ethanol or methanol at a temperature range between −20° C. and reflux. A suitable base may be an alkaline earth metal hydroxide such as lithium hydroxide, potassium hydroxide or sodium hydroxide. A suitable acid may be trifluoroacetic acid.

(x) alkylation of an amine of formula XX, wherein C is an optionally substituted aryl and R¹² is alkyl or hydrogen, with a suitable halide of formula XIX, wherein B is an optionally substituted aryl and halo is chloro, bromo or iodo, to form a compound of formula XXI.

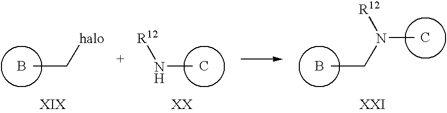

(xi) hydrolysis of a compound of formula XXII, wherein is B is an optionally substituted aryl and R¹³ is alkyl, to form a compound of formula XXIII.

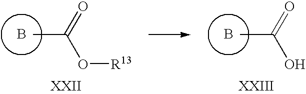

The reaction may be carried out with either base or acid in a suitable solvent such as water, tetrahydrofuran, ethanol or methanol at a temperature range between −20° C. and reflux. A suitable base may be an alkaline earth metal hydroxide such as lithium hydroxide, potassium hydroxide or sodium hydroxide. A suitable acid may be trifluoroacetic acid.

### Methods of Preparation of End Products

Another object of the invention are processes a, b, c, d, e or f, for the preparation of compounds of general Formula I, wherein P, Q, R², R³, R⁴, R⁵, R⁶ and R⁷, unless otherwise specified, are defined as hereinbefore, and salts thereof. When it is desired to obtain the acid salt, the free base may be treated with an acid such as a hydrogen halide such as hydrogen chloride in a suitable solvent such as tetrahydrofuran, diethyl ether, methanol, ethanol, chloroform or dichloromethane or mixtures thereof and the reaction may occur between −30° C. to +50° C.

These processes comprise,

(a) conversion of a compound of formula X, wherein R² is as defined above and R⁵ is an optionally substituted aryl or heteroaryl, to a compound of formula I, wherein Q is CONR⁴R⁵ as defined above, R⁴ is hydrogen, R⁵ is an optionally substituted aryl or heteroaryl and m is 0.

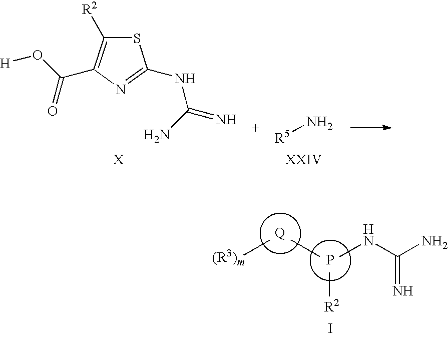

The amidation according to process (a) may be carried out by treating a compound of formula X with an appropriate amine such as a compound of formula XXIV, wherein R⁵ is an optionally substituted aryl or heteroaryl. The reaction can be performed neat or using a suitable solvent such as N,N-dimethylformamide, N,N-dimethylacetamide, dichloromethane or ethyl acetate at a temperature ranging from −25° C. to 150° C. The reaction may be aided by using a base such as potassium carbonate, triethylamine, N,N-diisopropylethylamine or 1,8-diazabicyclo[5.4.0]undec-7-ene or an acid such as trimethylaluminum or p-toluenesulfonic acid;

or,

the amidation of a compound of formula X, may be performed by activation by treating the compound of formula X with coupling reagents such as O-benzotriazol-1-yl-N,N,N,N′-tetramethyluronium tetrafluoroborate and N,N-diiospropylethylamine, 1-ethyl-3-(3-dimethylaminopropyl)carbodiimide hydrochloride and 1-hydroxybenzotriazole hydrate, 1,3-dicyclohexylcarbodiimide and 1-hydroxybenzotriazole hydrate, 1,1′-carbonyldiimidazole or O-(7-azabenzotriazol-1-yl)-N,N,N′,N′-tetramethyluronium hexafluorophosphate or using an acyl halide reagent such as oxalyl chloride, thionyl chloride or bromotrispyrrolidinophosphonium hexafluorophosphate followed by treatment with the appropriate amine such as a compound of formula XXIV. The reaction may be performed neat or using a suitable solvent such as N,N-dimethylformamide, N,N-dimethylaniline, dichloromethane or ethyl acetate at a temperature ranging from −25° C. to 150° C.

(b) conversion of a compound of formula XVI, wherein R² is as defined above, to a compound of formula I, wherein R³ is NR⁴COR⁵ as defined above and Q is an optionally substituted aryl.

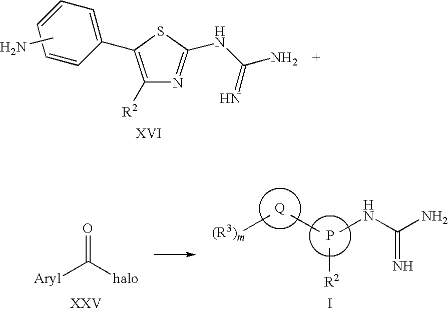

The amidation according to process (b) may be carried out by treating a compound of formula XVI with the appropriate acyl halide such as a compound of formula XXV wherein halo represents a halogen such as chlorine, fluorine or bromine. The reaction can be performed neat or using a suitable solvent such as N,N-dimethylformamide, dichloromethane or ethyl acetate at a temperature ranging from −25° C. to 150° C. The reaction may be aided by using a suitable base such as triethylamine, potassium carbonate, or 1,8-diazabicyclo[5.4.0]undec-7-ene or an acid such as trimethylaluminum or p-toluenesulfonic acid;

(c) conversion of a compound of formula XVI, wherein R² is as defined above, to a compound of formula I, wherein R³ is NR⁴COR⁵ as defined above and Q is an optionally substituted aryl.

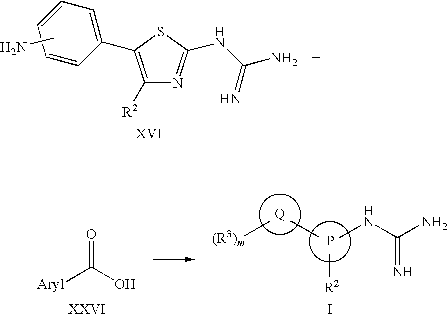

The amidation according to process (c) may be carried out by treating a compound of formula XVI with the appropriate carboxylic acid such as a compound of formula XXVI. The reaction can be performed neat or using a suitable solvent such as N,N-dimethylformamide, N,N-dimethylacetamide, dichloromethane or ethyl acetate at a temperature ranging from −25° C. to 150° C. The reaction may be aided by using a base such as potassium carbonate, triethylamine, N,N-diisopropylethylamine or 1,8-diazabicyclo[5.4.0]undec-7-ene or an acid such as trimethylaluminum or p-toluenesulfonic acid;

or,

the amidation of a compound of formula XVI, may be performed by activation by treating the compound of formula XXVI with coupling reagents such as O-benzotriazol-1-yl-N,N,N′,N′-tetramethyluronium tetrafluoroborate and N,N-diiospropylethylamine, 1-ethyl-3-(3-dimethylaminopropyl)carbodiimide hydrochloride and 1-hydroxybenzotriazole hydrate, 1,3-dicyclohexylcarbodiimide and 1-hydroxybenzotriazole hydrate, 1,1′-carbonyldiimidazole or O-(7-azabenzotriazol-1-yl)-N,N,N′,N′-tetramethyluronium to hexafluorophosphate or using an acyl halide reagent such as oxalyl chloride, thionyl chloride or bromotrispyrrolidinophosphonium hexafluorophosphate followed by treatment with the appropriate amine such as a compound of formula XVI. The reaction may be performed neat or using a suitable solvent such as N,N-dimethylformamide, N,N-dimethylaniline, dichloromethane or ethyl acetate at a temperature ranging from −25° C. to 150° C.

(d) conversion of a compound of formula XVI, wherein R² is as defined above, to obtain a compound of formula I wherein R³ is NR⁴(SO₂)R⁵ as defined above and Q is an optionally substituted aryl.

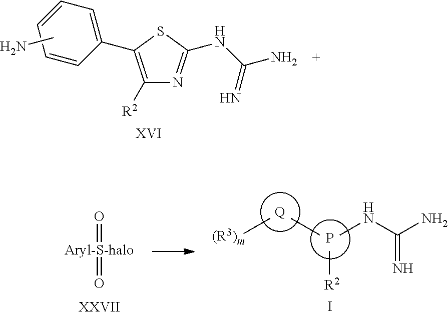

The reaction of process (d) may be carried out by treating a compound of formula XVI with the appropriate sulfonyl halide such as a compound of formula XXVII, wherein halo represents a halogen such as chlorine, fluorine, bromine or iodine. The reaction can be performed neat or using a suitable solvent such as tetrahydrofuran, methanol or water at temperatures in the range of 0° C. and 80° C. with or without a suitable base such as pyridine, triethylamine, sodium hydroxide or potassium carbonate.

(e) reductive amination of a compound of formula XVI, wherein R² is as defined above, to form a compound of formula I, wherein R³ is NR⁴R⁵ as defined above and Q is an optionally substituted aryl.

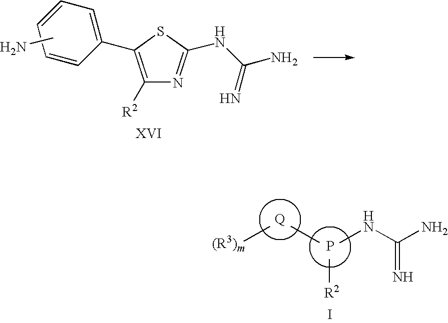

The reductive amination of process (e) may be carried out by treating a compound of formula XVI with an appropriate aldehyde or ketone using an appropriate reducing agent such as sodium cyanoborohydride, polystyrene supported cyanoborohydride, sodium triacetoxyborohydride, sodium borohydride, or in the presence of hydrogen or a hydrogenation catalyst. The reaction may be carried out in an appropriate solvent such as dichloromethane, methanol, ethanol, tetrahydrofuran or acetic acid at a temperature between −78° C. and reflux.

(f) reaction of a compound of formula IX, wherein R² is as defined above, with a suitable thiourea such as a compound of formula XXVIII wherein R¹⁴ is NHAryl or heteroaryl, to form a compound of formula I, wherein Q is an optionally substituted heteroaryl or aryl and R³ is NR⁴R⁵ as defined above and Q is an optionally substituted aryl.

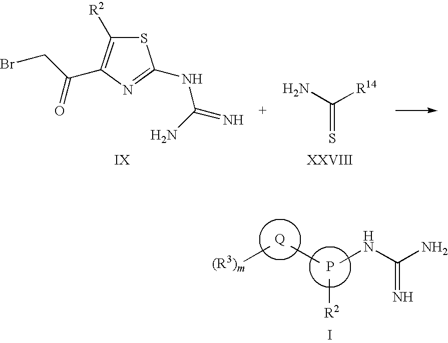

The reaction of process (f) may be carried by treating IX with XXVIII in a suitable solvent such as methanol, ethanol, isopropanol, acetone, acetonitrile or N,N-dimethylformamide at a temperature between −78° C. and reflux.

### EXAMPLES

Below follows a number of non-limiting examples of compounds of the invention.

### General Methods

Starting materials used were available from commercial sources, or prepared according to literature procedures.

¹H NMR spectra were recorded in the indicated deuterated solvent at 500 MHz, using a Varian INOVA 500 NMR spectrometer or a Bruker Advance 500 MHz spectrometer, or at 400 MHz, using a Bruker DPX400, or at 300 MHz, using a Bruker DPX300. Chemical shifts are given in ppm. Resonance multiplicities are denoted s, d, t, q, m and br for singlet, doublet, triplet, quartet, multiplet, and broad respectively.

HPLC assays were performed using an Agilent 1100 Series system equipped with a Synergi MAX RP, C12-column, (4 μm, 50×3.0 mm). The mobile phase system consisted of A: aqueous buffer pH 4, containing 5 mM formic acid and 5 mM ammonium formate and B: acetonitrile. A linear gradient was applied running from 5% to 50% B in 3 min followed by 50-95% B in 1.5 min. Or HPLC assays were preformed using a Agilent 1100 or a Waters Alliance HT (2790 & 2795) system equipped with a Phemonenex Gemini C18 (5 μm, 50×2 mm). The mobile phase used was varying gradients of water, acetonitrile and 1% formic acid in water/acetonitrile (50:50).

Mass spectra (MS) were recorded on either a Micromass ZQ single quadrupole or a Micromass Quattro micro, both equipped with a pneumatically assisted electrospray interface, or mass spectra (MS) were recorded on ZQ ESCi or Waters ZDM ESCi. Alternatively, Mass spectra (MS) data was generated on an LCMS system where the HPLC component comprised of a Waters Alliance HT (2790 or 2795) equipment and was run on a Phemonenex Gemini C18 5 μm, 50×2 mm column (or similar) eluting with a basic eluent (using a 4.5 min gradient between 0-95% water/acetonitrile with 5% of a 0.1% 880 ammonia in water/acetonitrile 50:50 mixture); and the MS component comprised generally of a Waters ZQ mass spectrometer scanning over an appropriate mass range. Chromatograms for Electrospray (ESI) (positive and negative), Base Peak Intensity, and UV Total Absorption from 220-300 nm, were generated and values for m/z are given; generally, only ions which indicate the parent mass are reported and unless otherwise stated the value quoted is the [M+H]⁺ for positive ion mode.

Preparative HPLC was performed using Waters Fraction Lynx Purification System using, A: Kromasil C8 (20×100 mm, 5 μm), or B: Waters Sunfire C18 5 mm 19×100 mm columns. The mobile phase used was varying gradients of 0.1 M ammonium acetate buffer and acetonitrile. The flow was 30 mL/min. MS triggered fraction collection was used. Mass spectra were recorded on a Micromass Quattro micro, equipped with pneumatically assisted electrospray interface.

Column chromatography was performed using Merck Silica gel 60 (0.040-0.063 mm).

Compounds have been named using ACD/Name, version 8.0 or 9.0, software from Advanced Chemistry Development, Inc. (ACD/Labs), Toronto ON, Canada, www.acdlabs.com, 2004.

### Example 1

### tert-Butyl{[(aminocarbonothioyl)amino](imino)methyl}carbamate

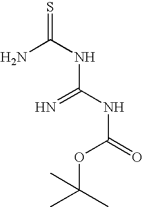

A solution of di-tert-butyl dicarbonate (100 g, 458 mmol) in N,N-dimethylformamide (50 mL) was added dropwise over 1 h to a mixture of 1-[amino(imino)methyl]thiourea (50 g, 424 mmol) and 4-dimethylaminopyridine (0.450 g, 3.68 mmol) in N,N-dimethylformamide (100 mL). After complete addition the mixture was stirred for 20 h at 35° C. The mixture was concentrated in vacuo and the residue was manually shaken with water (70 mL).

Within 1 min the residue solidified. Shaking was continued for 6 min, and the mixture was kept at 4° C. for 1 h. The precipitate was collected by filtration, washed with cold water (30 mL) and dried in vacuo to afford 73.39 g (80% yield) of the title compound: ¹³C NMR (125.7 MHz, DMSO-d₆): δ 162.98, 157.70, 156.36, 79.93, 28.50; MS (ESI) m/z 219.2 [M+1]⁺

### Example 2

### Ethyl 2-({[(tert-butoxycarbonyl)amino](imino)methyl}amino)-1,3-thiazole-4-carboxylate hydrobromide

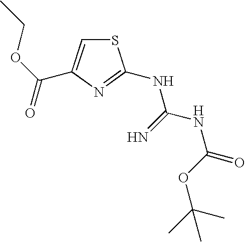

A solution of 90% ethyl bromopyruvate (24.6 g, 15.8 mL, 113.5 mmol) in acetone (30 mL) was added dropwise over 1 h to an ice cooled stirred suspension of tert-butyl {[(aminocarbonothioyl)amino](imino)methyl}carbamate in acetone (80 mL). After complete addition the reaction mixture was stirred at room temperature for an additional 16 h and was then cooled at 4° C. for 2 h. The precipitate was collected by filtration, washed with a small amount of cold acetone, and dried in vacuo to give 22.32 g. Another 1.44 g were collected from the combined filtrates after addition of an equal volume of hexane and cooling to 4° C. for 4 h to afford in total 23.76 g (71% yield) of the title compound: ¹H NMR (500 MHz, DMSO-d₆): δ 9.5-8.4 (m, 3H), 8.05 (s, 1H), 4.26 (q, 2H), 1.48 (s, 9H), 1.28 (t, 3H), (major conformer); ¹³C NMR (125.7 MHz, DMSO-d₆): δ 176.39, 167.07, 160.73, 152.94, 141.51, 123.80, 83.67, 61.53, 28.36, 14.84; MS (ESI) m/z 315.1 [M+1]⁺

### Example 3

### 2-({[(tert-Butoxycarbonyl)amino](imino)methyl}amino)-1,3-thiazole-4-carboxylic acid

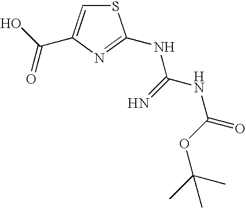

A solution of lithium hydroxide monohydrate (5.61 g, 133 mmol) in water (90 mL) was added to a stirred suspension of ethyl 2-({[(tert-butoxycarbonyl)amino](imino)methyl}amino)-1,3-thiazole-4-carboxylate hydrobromide (22 g, 55.7 mmol) in tetrahydrofuran (180 mL). The resulting emulsion was stirred vigorously at room temperature for 16 h. Additional lithium hydroxide monohydrate (4.2 g, 100 mmol) was added and stirring was continued for 6 h at 35-40° C. The tetrahydrofuran was evaporated and the mixture was acidified by addition of 27% citric acid (70 mL). After stirring for 30 min at room temperature the precipitate was collected by filtration, washed with water and dried in vacuo to afford 15.9 g (99% yield) of the title compound: ¹H NMR (500 MHz, DMSO-d₆): δ 10.38 (br s, 1H), 9.27 (br s, 1H), 8.42 (br s, 1H), 7.22 (br s, 1H), 1.46 (s, 9H); MS (ESI) m/z 287.1 [M+1]⁺

### Example 4

### N-[4-Ethyl-5-(4-nitrophenyl)-1,3-thiazol-2-yl]guanidine

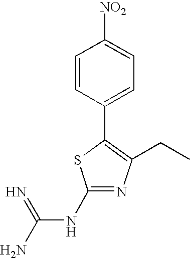

To a cooled (0° C.) stirred suspension of 1-nitro-4-(2-nitro-vinyl)-benzene (3.50 g, 15.75 mmol; described in: Demicheli G. et al. Tetrahedron Lett. 2001, 42(12), 2401-2403) in methanol (30 mL) was added aqueous hydrogen peroxide (5.84 mL, 66.15 mmol, 35 wt %) followed by aqueous sodium hydroxide (2.75 mL, 5.50 mmol). The reaction mixture was stirred at 0° C. for approximately 1 h and at room temperature for 1 h. The reaction mixture was poured into ice/water (100 mL), acidified to pH 1 with hydrochloric acid (2 M) and the aqueous layer was extracted with ethyl acetate (3×50 mL). The combined organic phases were washed with saturated aqueous sodium bicarbonate (100 mL), water (100 mL), sodium thiosulphate (2 M, 100 mL), and brine (100 mL), dried over magnesium sulfate and concentrated in vacuo to give 2-ethyl-2-nitro-3-(4-nitrophenyl)oxirane as a yellow solid. The crude 2-ethyl-2-nitro-3-(4-nitrophenyl)oxirane was suspended in ethanol (30 mL), 1-[amino(imino)methyl]thiourea (2.8 g, 23.63 mmol) was added and the reaction was heated to 90° C. for 2 h. The reaction was allowed to cool to room temperature, and the ethanol was removed in vacuo. The residue was taken up in ethyl acetate (200 mL) and washed with water (200 mL). The water was further extracted with ethyl acetate (3×100 mL) and the organic phases and the formed solids were combined and concentrated in vacuo to afford 4.47 g, (97% yield) of the title compound: ¹H NMR (300 MHz, DMSO-d₆) δ 8.22 (d, 2H), 7.57 (d, 2H), 7.04 (s, 4H), 2.68 (q, 2H), 1.24 (t, 3H); MS (ESI) m/z 292.45 [M+H]⁺.

### Example 5

### N-[5-(4-Aminophenyl)-4-ethyl-1,3-thiazol-2-yl]guanidine

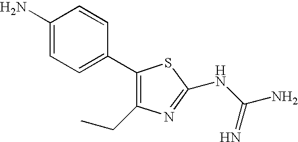

To a stirred suspension of N-[4-ethyl-5-(4-nitrophenyl)-1,3-thiazol-2-yl]guanidine (2.00 g, 7.65 mmol) and palladium on carbon (10 mol %, 0.600 g) in anhydrous methanol (20 mL) was added anhydrous ammonium formate (2.00 g, 35.19 mmol) in one portion. The reaction was heated at 140° C. for 5 min in a sealed 20 mL microwave vessel. The palladium was removed by filtration, washed with methanol (100 mL) and the filtrates were concentrated in vacuo. The residue was dry loaded onto silica and purified by column chromatography (using a gradient 0-10% ammonia (7 M) in methanol/dichloromethane as the eluent) to afford 1.184 g, (66% yield) of the title compound: ¹H NMR (300 MHz, DMSO-d₆) δ 6.98 (d, 2H), 6.78 (s, 1H), 6.57 (d, 2H), 5.15 (s, 2H), 1.16 (t, 3H); MS (ESI) m/z 262.50 [M+H]⁺.

### Example 6

### N-[4-Methyl-5-(3-nitrophenyl)-1,3-thiazol-2-yl]guanidine

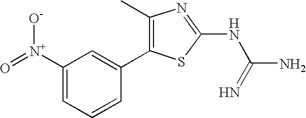

Sodium hydroxide (2 M, 19 mL, 38.4 mmol) and aqueous hydrogen peroxide (27 mL, 307 mmol, 35 wt %) was added to a cooled (0° C.) suspension of 1-nitro-3-[(1Z)-2-nitroprop-1-en-1-yl]benzene (16 g, 76.9 mmol; described in: Karmarkar S. N. et al. Synthesis 1985, 5, 510-512) in methanol (190 mL). The resulting mixture was allowed to warm to room temperature and stirred for 4 h. The reaction mixture was poured into 1000 mL ice/water. The mixture was acidified to approximately pH 1 using hydrochloric acid (2 M) and extracted with diethyl ether. The combined organic phases were washed with water, aqueous bicarbonate solution, sodium metabisulfite (2 M), and brine, dried over magnesium sulfate and the solvent was evaporated to give the crude 2-methyl-2-nitro-3-(3-nitrophenyl)oxirane. 1-[Amino(imino)methyl]thiourea (9.09 g, 76.9 mmol) was dissolved in methanol (600 mL) and this solution was added to the crude 2-methyl-2-nitro-3-(3-nitrophenyl)oxirane. The reaction was stirred over night and a precipitate formed. The reaction mixture was concentrated to approximately half the volume and filtered to afford 13.06 g (61% yield) of the title compound: MS (ESI) m/z 278.52 [M+H]⁺.

### Example 7

### N-[5-(3-Aminophenyl)-4-methyl-1,3-thiazol-2-yl]guanidine

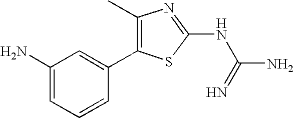

Palladium on carbon (10 mol %, 0.36 g) was added to a suspension of N-[4-methyl-5-(3-nitrophenyl)-1,3-thiazol-2-yl]guanidine (1.20 g, 4.33 mmol) in methanol (50 mL) under an atmosphere of argon. The reaction was stirred vigorously over night under an atmosphere of hydrogen. The reaction was filtered and concentrated in vacuo to afford 0.851 g (79% yield) of the title compound: ¹H NMR (300 MHz, DMSO-d₆) δ 7.01 (t, 1H), 6.85 (br s, 4 H), 6.58 (m, 1H), 6.50 (br d, 1H), 6.45 (br d, 1H), 5.15 (br s, 2H), 2.25 (s, 3H); MS (ESI) m/z 248.48 [M+H]⁺.

### Example 8

### Methyl 4-[(1E)-2-nitroprop-1-en-1-yl]benzoate

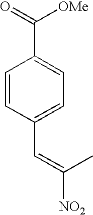

Methyl 4-formylbenzoate (1.64 g, 10 mmol), nitroethane (2.3 mL, 32 mmol) and ammonium acetate (924 mg, 12 mmol) were dissolved in acetic acid (9 mL) and the resulting solution was heated at reflux for 2 h. The reaction was cooled to ambient temperature and poured into ice/water (50 mL). Ethyl acetate (50 mL) was added and the organic phase was washed with water (2×50 mL) and brine (50 mL), dried over magnesium sulfate and concentrated in vacuo. Recrystallisation from boiling ethyl acetate/isohexane (10:1, 10 mL) gave 1.13 g (51% yield) of the title compound: ¹H NMR (400 MHz, CDCl₃) δ 8.10 (m, 3H), 7.45 (d, 2H), 4.05 (s, 3H), 2.55 (s, 3H).

### Example 9

### Methyl 4-(2-{[amino(imino)methyl]amino}-4-methyl-1,3-thiazol-5-yl)benzoate

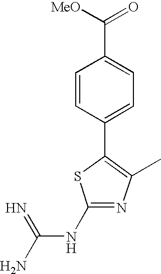

Methyl 4-[(1E)-2-nitroprop-1-en-1-yl]benzoate (186 mg, 0.84 mmol) was suspended in methanol (2 mL) and cooled in an ice bath. Aqueous hydrogen peroxide (310 μL, 35 wt %) and sodium hydroxide (2 M, 150 μL) were added and the reaction stirred at ambient temperature for 3.5 h. The reaction mixture was poured into ice/water (20 mL), acidified to pH 1 with hydrochloric acid (2 M) and extracted with diethyl ether (2×20 mL). The combined organic phases were washed with water (30 mL), aqueous bicarbonate (30 mL), sodium metabisulfate (2 M, 30 mL) and brine (30 mL), dried over magnesium sulfate and concentrated in vacuo to afford methyl 4-(3-methyl-3-nitrooxiran-2-yl)benzoate (137 mg, 0.58 mmol, 69% yield). Methyl 4-(3-methyl-3-nitrooxiran-2-yl)benzoate (132 mg, 0.56 mmol) and 1-[amino(imino)methyl]thiourea (100 mg, 0.84 mmol) were suspended in ethanol (4 mL) and heated at reflux for 14 h. The formed precipitate was filtered off, washed with ethanol (2×5 mL) and dried on the filter to afford 0.110 g (68% yield) of the title compound: ¹H NMR (300 MHz, DMSO-d₆) δ 8.07 (d, 2H), 7.51 (d, 2H), 7.05 (br s, 4H), 3.72 (s, 3H), 2.35 (s, 3H); MS (ESI) m/z 291.58 [M+H]⁺.

### Example 10

### 4-(2-{[Amino(imino)methyl]amino}-4-methyl-1,3-thiazol-5-yl)benzoic acid

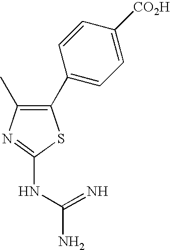

Aqueous lithium hydroxide (1 M, 5 mL) was added to a solution of methyl 4-(2-{[amino(imino)methyl]amino}-4-methyl-1,3-thiazol-5-yl)benzoate (499 mg, 1.72 mmol) in tetrahydrofuran (5 mL), and the reaction mixture was stirred for 2 h at 50° C. The reaction was cooled to room temperature and the tetrahydrofuran was removed in vacuo. The aqueous phase was acidified to pH 1 by slow addition of hydrochloric acid (2 M) under vigorous stirring. The formed precipitate was filtered off, washed with water (3×20 mL) and dried in the vacuum oven for 8 h (65° C.) to afford 0.370 g (78% yield) of the title compound. ¹H NMR (300 MHz, DMSO-d₆) δ 12.86 (br s, 1H), 8.21 (s, 4H), 8.01 (d, 2H), 7.65 (d, 2H), 2.42 (s, 3H); MS (ESI) m/z 277.6 [M+11]⁺.

### Example 11

### N-(5-Acetyl-4-methyl-1,3-thiazol-2-yl)guanidine

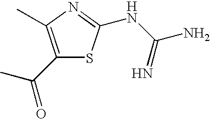

Bromine (4.87 mL, 95 mmol) was added to a solution of sodium bromide (39.96 g, 388 mmol) in water (150 mL). This mixture was thoroughly mixed and added dropwise over 40 min to a cooled (0° C.) suspension of 2,4-pentanedione (10.26 mL, 100 mmol) in aqueous sodium hydroxide (0.25 M, 500 mL). Once the addition was complete, aqueous hydrogen bromide (1 M, 35 mL) was added. The aqueous phase was extracted with diethyl ether and the combined organic phases were washed with brine, dried over magnesium sulfate and the solvent was evaporated in vacuo. The residue was dissolved in ethanol (20 mL) and added to a refluxing solution of 1-[amino(imino)methyl]thiourea (8.85 g, 75 mmol) in to ethanol (200 mL). The reaction mixture was refluxed for 1 h, cooled and filtered to afford 5.20 g of the title compound as the hydrobromide. The filtrate was evaporated and the crude product was purified by column chromatography, using a gradient of 3-7% methanol in dichloromethane containing 1% ammonia, to afford 0.371 g of the title compound: MS (ESI) m/z 199 [M+H]⁺

### Example 12

### N-[5-(Bromoacetyl)-4-methyl-1,3-thiazol-2-yl]guanidine

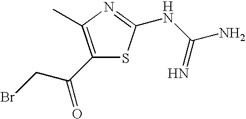

A solution of bromine (1.356 mL, 26.23 mmol) in glacial acetic acid (12 mL) was added dropwise over 45 min to a suspension of N-(5-acetyl-4-methyl-1,3-thiazol-2-yl)guanidine (5.194 g, 26.23 mmol) in glacial acetic acid (65 mL) and water (10 mL) at 80° C. The orange suspension was stirred at 80° C. for 1 h, cooled and filtered to afford 3.914 g (54% yield) of the title compound: MS (ESI) m/z 277 and 279 [M+H]⁺

### Example 13

### 2-{[Amino(imino)methyl]amino}-N-1-anthryl-1,3-thiazole-4-carboxamide

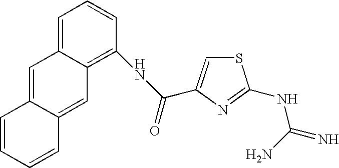

A mixture of 1-ethyl-3-(3-dimethylaminopropyl)carbodiimide hydrochloride (71 mg, 0.37 mmol) and 1-aminoanthracene (64 mg, 0.33 mmol) were added to a suspension of 2-({[(tert-butoxycarbonyl)amino](imino)methyl}amino)-1,3-thiazole-4-carboxylic acid (94 mg, 0.33 mmol) in dichloromethane (4 mL). The mixture was stirred at room temperature for 21 h. Dichloromethane (10 mL) was added and the mixture was washed three times with 10% citric acid, three times with an aqueous sodium bicarbonate solution, and with brine. After drying over sodium sulfate the solvent was evaporated in vacuo and the residue was purified by column chromatography, using dichloromethane/methanol (30:1) as the eluent. 18 mg of this intermediate were dissolved in 1.5 mL of trifluoroacetic acid. After 45 min the reaction mixture was concentrated in vacuo to afford 18 mg (15% yield) of the title compound: ¹HNMR (500 MHz, DMSO-d₆): δ 12.62 (br s, 1H), 10.80 (s, 1H), 8.67-8.55 (m, 4H), 8.12-8.07 (m, 3H), 7.60-7.50 (m, 3H), 5.0 (br s, 4H); ¹³CNMR (125.7 MHz, DMSO-d₆): δ 160.75, 159.99, 159.78, 154.22, 146.22, 133.75, 132.51, 131.91, 131.74, 129.15, 128.58, 127.79, 127.34, 126.64, 125.71, 124.26, 122.51, 120.55; MS (ESI) m/z 362.1 [M+1]⁺

### Example 14

### 2-{[Amino(imino)methyl]amino}-N-(9-ethyl-9H-carbazol-3-yl)-1,3-thiazole-4-carboxamide

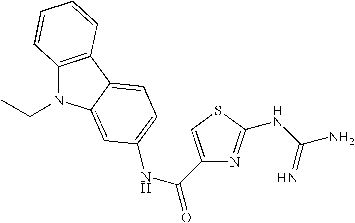

3-Amino-9-ethylcarbazole was delivered preweighed (0.1 mmol) under an atmosphere of argon in a 4 mL round bottom vial. N,N-Dimethylformamide (1 mL) was added and the amine was shaken for 15 min. 1 mL of a stock solution, made of 2-({[(tert-butoxycarbonyl)amino](imino)methyl}amino)-1,3-thiazole-4-carboxylic acid (6.871 g), O-benzotriazol-1-yl-N,N,N′,N′-tetramethyluronium tetrafluoroborate (9.244 g) and N,N-diisopropylethylamine (8.34 mL) in dry N,N-dimethylformamide (231.6 mL) to give a total volume of 240 mL, was added. The vial was capped and shaken for 16 h. The reaction mixture was evaporated using a vacuum centrifuge (Genevac HT-12). Trifluoroacetic acid (400 μL) was added to the glass vial, and the vial was shaken for 2 h. The reagent was reduced using a vacuum centrifuge (Genevac HT-12). The crude mixture was purified by preparative HPLC to afford 22.6 mg of the title compound (60% yield from 2-({amino[(tert-butoxycarbonyl)imino]methyl}amino)-1,3-thiazole-4-carboxylic acid): MS (ESI) m/z 379.3 [M+1]⁺

### Example 15

### N-[3-(2-{[Amino(imino)methyl]amino}-4-methyl-1,3-thiazol-5-yl)phenyl]-4-(benzyloxy)benzamide

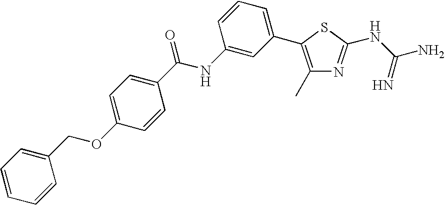

A solution of N-[5-(3-aminophenyl)-4-methyl-1,3-thiazol-2-yl]guanidine (24.7 mg, 0.1 mmol) in anhydrous tetrahydrofuran (2 mL) and triethylamine (30.4 mg, 0.3 mmol) was prepared. This solution was added to a 4 mL glass vial containing 4-(benzyloxy)benzoyl chloride (27.1 mg, 0.11 mmol). The vial was capped and shaken over night. The solution was concentrated in vacuo and the residue dissolved in dimethylsulfoxide and the crude product was purified by preparative HPLC and concentrated in vacuo to afford 18.4 mg (40% yield) of the title compound: MS (ESI) m/z 458.3 [M+H]⁺

### Example 16

### N-[3-(2-{[Amino(imino)methyl]amino}-4-methyl-1,3-thiazol-5-yl)phenyl]-4′-methoxybiphenyl-4-sulfonamide

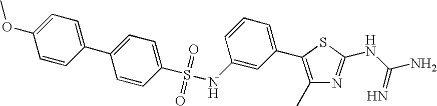

A solution of N-[5-(3-aminophenyl)-4-methyl-1,3-thiazol-2-yl]guanidine (24.7 mg, 0.1 mol) in anhydrous tetrahydrofuran (2 mL) and pyridine (47.5 mg, 0.6 mmol) was prepared. This solution was added to a 4 mL glass vial containing 4′-methoxybiphenyl-4-sulfonyl chloride (31.1 mg, 0.11 mmol). The vial was capped and shook over night. The solution was concentrated in vacuo and the residue was dissolved in dimethylsulfoxide and the crude product was purified by preparative HPLC to afford 51.0 mg (quantitative yield) of the title compound: MS (ESI) m/z 494.3 [M+H]⁺

### Example 17

### N-[3-(2-{[Amino(imino)methyl]amino}-4-methyl-1,3-thiazol-5-yl)phenyl]-4-(1,2,3-thiadiazol-4-yl)benzamide

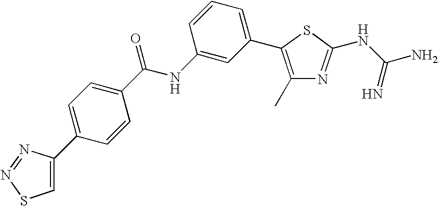

The title compound was synthesized as described for N-[3-(2-{[amino(imino)methyl]amino}-4-methyl-1,3-thiazol-5-yl)phenyl]-4-(benzyloxy)benzamide in 21% yield starting from 4-(1,2,3-thiadiazol-4-yl)benzoyl chloride: MS (ESI) m/z 436.2 [M+H]⁺.

### Example 18

### N-[3-(2-{[Amino(imino)methyl]amino}-4-methyl-1,3-thiazol-5-yl)phenyl]-3-chloro-6-ethyl-1-benzothiophene-2-carboxamide

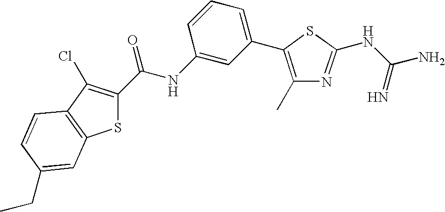

The title compound was synthesized as described for N-[3-(2-{[amino(imino)methyl]amino}-4-methyl-1,3-thiazol-5-yl)phenyl]-4-(benzyloxy)benzamide in 26% yield starting from 3-chloro-6-ethyl-1-benzothiophene-2-carbonyl chloride: MS (ESI) m/z 470.2 [M+H]⁺.

### Example 19

### N-[3-(2-{[Amino(imino)methyl]amino}-4-methyl-1,3-thiazol-5-yl)phenyl]-2-naphthamide

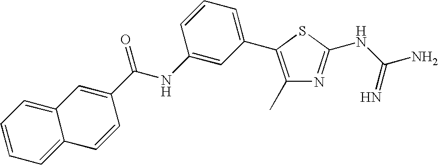

The title compound was synthesized as described for N-[3-(2-{[amino(imino)methyl]amino}-4-methyl-1,3-thiazol-5-yl)phenyl]-4-(benzyloxy)benzamide in 92% yield starting from 2-naphthoyl chloride: MS (ESI) m/z 402.5 [M+H]⁺.

### Example 20

### Benzyl 4-({[4-(2-{[amino(imino)methyl]amino}-4-ethyl-1,3-thiazol-5-yl)phenyl]amino}methyl)piperidine-1-carboxylate

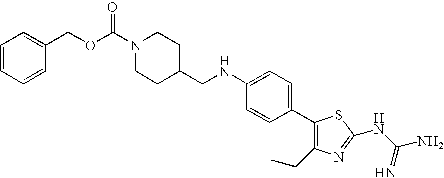

To a stirred solution of N-[5-(4-aminophenyl)-4-ethyl-1,3-thiazol-2-yl]guanidine (150 mg, 0.57 mmol) and benzyl 4-formylpiperidine-1-carboxylate (170 mg, 0.69 mmol) in dichloromethane (10 mL) was added sodium triacetoxyborohydride (243 mg, 1.15 mmol) and the resulting solution was stirred at room temperature under an atmosphere of nitrogen for 2 h. The dichloromethane was removed in vacuo and the residue taken up in ethyl acetate (50 mL). The organic phase was washed with water (50 mL) and brine (50 mL), dried over magnesium sulfate and concentrated in vacuo. The residue was purified by column chromatography (using a gradient 0-10% methanol (basic) in dichloromethane as the eluent) to afford 0.221 g (79% yield) of the title compound: ¹H NMR (300 MHz, DMSO-d₆) δ 7.42-7.28 (m, 5H), 7.02 (d, 2H), 6.81 (s, 4H), 6.58 (d, 2H), 5.81 (t, 1H), 5.07 (s, 2H), 4.03 (d, 2H), 2.92 (t, 2H), 2.87-2.68 (m, 2H), 1.76 (d, 2H), 1.16 (t, 3H), 1.13-1.01 (m, 3H); MS (ESI) m/z 493.66 [M+H]⁺.

### Example 21

### N″-{5-[4-(Dibenzylamino)phenyl]-4-ethyl-1,3-thiazol-2-yl}guanidine

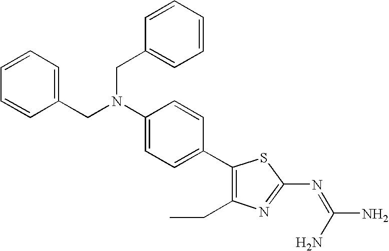

A solution of N-[5-(4-aminophenyl)-4-ethyl-1,3-thiazol-2-yl]guanidine (105 mg, 0.40 mmol), benzaldehyde (0.049 mL, 0.48 mmol) and acetic acid (0.4 mL) in dichloromethane (4 mL) was stirred at room temperature for 30 min and then polysyrene supported cyanoborohydride (244 mg, 1.00 mmol) was added. After approx 1 h the reaction was complete. The polymer-supported cyanoborohydride was filtered off and washed with dichlorormethane (20 mL). The filtrate was concentrated in vacuo, redissolved in methanol (5 mL) and loaded onto a 5 g SCX cartridge, washed with methanol, water and acetonitrile to remove excess benzaldehyde and acetic acid. The product was eluted with methanol/ammonium hydroxide (9:1), and concentrated in vacuo to afford a solid (approx 130 mg). This was dissolved in dimethylsulfoxide and purified by preparative HPLC to afford 12.5 mg (7% yield) the title compound: ¹H NMR (300 MHz, DMSO-d₆) δ 7.37-7.21 (m, 10H), 7.05 (d, 2H), 6.79 (s, 4H), 6.69 (d, 2H), 4.71 (s, 4H), 2.50 (q, 2H), 1.14 (t, 3H); MS (ESI) m/z 442.61 [M+H]⁺.

### Example 22

### N-(5-{3-[(2-Bromo-6-hydroxybenzyl)amino]phenyl}-4-methyl-1,3-thiazol-2-yl)guanidine

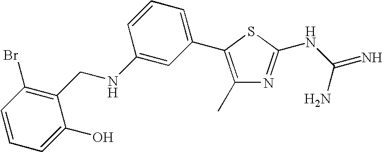

Polystyrene-supported cyanoborohydride (0.122 g, 0.5 mmol) was added to a stirred solution of N-[5-(3-aminophenyl)-4-methyl-1,3-thiazol-2-yl]guanidine (0.049 g, 0.2 mmol) and 2-bromo-6-hydroxybenzaldehyde (0.24 mmol) in dichloromethane/acetic acid (10:1, 4 mL) at room temperature. The resulting mixture was shaken at room temperature for 3 h, and the resin was removed by filtration. The reaction mixture was concentrated in vacuo, and purified by preparative HPLC to afford 11.1 mg (13% yield) of the title compound: MS (ESI) m/z 434.81 [M+H]⁺.

### Example 23

### N-[5-(4-{[4-(2-Hydroxyphenyl)piperazin-1-yl]carbonyl}phenyl)-4-methyl-1,3-thiazol-2-yl]guanidine

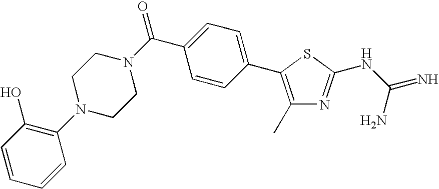

A solution of O-(7-azabenzotriazol-1-yl)-N,N,N′,N′-tetramethyluronium hexafluorophosphate (0.084 g, 0.22 mmol) in N,N-dimethylaniline (1 mL) was added to a mixture of 4-(2-{[amino(imino)methyl]amino}-4-methyl-1,3-thiazol-5-yl)benzoic acid, N,N-diidopropylethylamine (0.104 mL) and 1-(2-hydroxyphenyl)piperazine (0.045 g, 0.25 mmol) in N,N-dimethylaniline (1 mL). The resulting mixture was stirred at room temperature overnight and then concentrated in vacuo. The mixture was redissolved in 2% methanol in dichloromethane, washed with saturated aqueous sodium hydrogencarbonate and water, concentrated in vacuo and purified by preparative HPLC to afford 35.1 mg (47% yield) of the title compound: ¹H NMR (500 MHz, DMSO-d₆) δ 7.47-7.43 (m, 4H), 6.92-6.74 (m, 5H), 3.80-3.50 (m, 4H), 3.00-2.90 (m, 4H), 2.32-2.31 (s, 3H).

### Examples 24-30

A solution of N[5-(bromoacetyl)-4-methyl-1,3-thiazol-2-yl]guanidine (55.4 mg; 0.20 mmol) in ethanol (2 mL) was added to the appropriate thiourea (0.21 mmol) into 46-well Bhodan test-tubes. The reactions were stirred and heated at reflux for 1 h. The reactions were cooled and filtered to give the compounds listed in Table 1.

TABLE 1

| | | | | MS (ESI) |
|---|---|---|---|---|
| | | | Yield mg | m/z |
| Ex | Structure | Name | (%) | [M + H]⁺ |
| 24 | 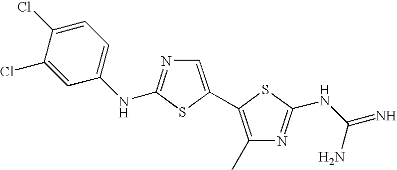 | N-{2′-[(3,4- dichlorophenyl)amino]-4- methyl-5,5′-bi-1,3-thiazol-2- yl}guanidine | 54 (68%) | 399.56 |
| 25 | 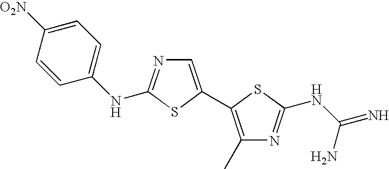 | N-{4-methyl-2′-[(4- nitrophenyl)amino]-5,5′-bi- 1,3-thiazol-2-yl}guanidine | 65 (87%) | 376.54 |
| 26 | 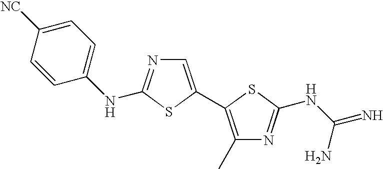 | N-{2′-[(4- cyanophenyl)amino]-4- methyl-5,5′-bi-1,3-thiazol-2- yl}guanidine | 70 (98%) | 356.48 |
| 27 | 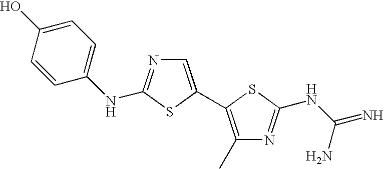 | N-{2′-[(4- hydroxyphenyl)amino]-4- methyl-5,5′-bi-1,3-thiazol-2- yl}guanidine | 27 (39%) | 347.50 |
| 28 | 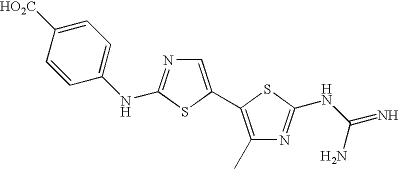 | 4-[(2′- {[amino(imino)methyl]amino}- 4′-methyl-5,5′-bi-1,3- thiazol-2-yl)amino]benzoic acid | 62 (83%) | 375.33 |
| 29 | 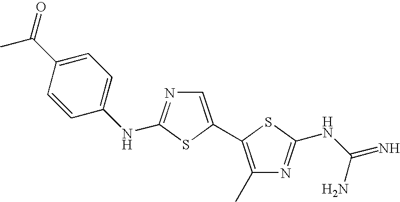 | N-{2′-[(4- acetylphenyl)amino]-4- methyl-5,5′-bi-1,3-thiazol-2- yl}guanidine | 68 (91%) | 373.52 |
| 30 | 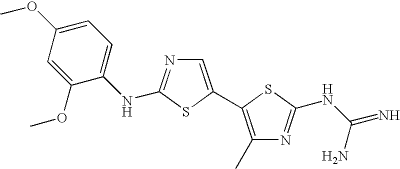 | N-{2′-[(2,4- dimethoxyphenyl)amino]-4- methyl-5,5′-bi-1,3-thiazol-2- yl}guanidine | 74 (95%) | 391.39 |

### Example 31

### 4-(2-{[Amino(imino)methyl]amino}-4-methyl-1,3-thiazol-5-yl)-N-(4-pyffolidin-1-ylbutyl)benzamide

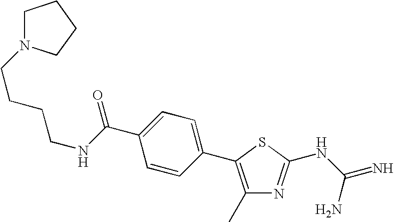

A solution of O-(7-azabenzotriazol-1-yl)-N,N,N′,N′-tetramethyluronium hexafluorophosphate (84 mg, 0.22 mmol) in N,N-dimethylacetamide (1 mL) was added to a mixture of 4-(2-{[amino(imino)methyl]amino}-4-methyl-1,3-thiazol-5-yl)benzoic acid (55 mg, 0.20 mmol), N,N-diisopropylethylamine (104 μL, 0.6 mmol) and 4-pyrrolidin-1-ylbutan-1-amine (36 mg, 0.25 mmol) in N,N-dimethylacetamide (1 mL). The resulting mixture was stirred at room temperature overnight and concentrated in vacuo. The residue was dissolved in 2% methanol in dichloromethane, washed with saturated aqueous sodium hydrogen carbonate and water and concentrated in vacuo. Purification by preparative reverse phase HPLC gave 24.4 mg (31% yield) of the title compound; ¹H NMR (500 MHz, DMSO-d₆) δ 8.43 (t, 1H), 7.84 (d, 2H), 7.43 (d, 2H), 6.91 (s, 3H), 2.45-2.41 (m, 8H), 2.31 (s, 3H), 1.70-1.65 (m, 4H), 1.59-1.54 (m, 2H), 1.52-1.46 (m, 2H; MS (ESI) m/z 401 [M+H]⁺

### Example 32

### Benzyl 4-{[(4-{2-[(diaminomethylene)amino]-4-methyl-1,3-thiazol-5-yl}phenyl)amino]methyl}piperidine-1-carboxylate

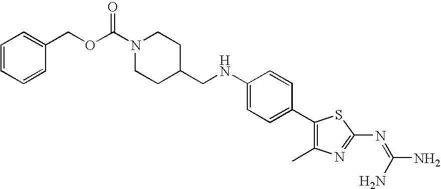

To a stirred solution of 2-[5-(4-aminophenyl)-4-methyl-1,3-thiazol-2-yl]guanidine (495 mg, 2.00 mmol, described in Alig L.; Edenhofer A.; Hilpert K.; Weller T. Preparation of thiazolecarboxamides as vitronectin receptor antagonists. EP 928790 A1, 19990714) and benzyl 4-formylpiperidine-1-carboxylate (594 mg, 2.40 mmol) in dichloromethane (20 mL) was added sodium triacetoxyborohydride (848 mg, 4.00 mmol) and the resulting solution stirred at room temperature under an atmosphere of nitrogen for 2 h. The solvent was removed in vacuo and the residue was taken up in ethyl acetate (100 mL). The organics were washed with water and brine, dried over sodium sulfate and concentrated in vacuo. The residue was purified by column chromatography, using 0-5% 7 M methanolic ammonia in dichloromethane as the eluent, to give 0.901 g (94% yield) of the title compound: ¹H NMR (300 MHz, DMSO-d₆) δ 7.28-7.40 (m, 5H), 7.05 (d, 2H), 6.78 (br s, 4H), 6.57 (d, 2H), 5.80 (t, 1H), 5.07 (s, 2H), 4.03 (d, 2H), 2.92 (t, 2H), 2.80 (br s, 2H), 2.18 (s, 3H), 1.76 (d, 3H), 1.02-1.14 (m, 2H); MS (ESI) m/z 479.58 [M+H]⁺.

### Example 33

### 2-(5-{4-[Benzyl(piperidin-4-ylmethyl)amino]phenyl}-4-methyl-1,3-thiazol-2-yl)guanidine

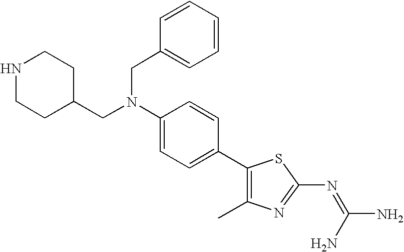

To a stirred suspension of benzyl 4-{[(4-{2-[(diaminomethylene)amino]-4-methyl-1,3-thiazol-5-yl}phenyl)amino]methyl}piperidine-1-carboxylate (0.900 g, 1.88 mmol) in acetonitrile (30 mL) was added trimethylsilyl iodide (0.8 mL, 5.64 mmol) and the resulting solution was stirred at room temperature under an atmosphere of nitrogen for 18 h. The reaction mixture was then loaded onto a 20 g SCX2 cartridge and washed with acetonitrile (60 mL) and methanol (60 mL) before the product was eluted with 7 N methanolic ammonia. Purification by column chromatography, using 0-10% methanolic ammonia in dichloromethane as the eluent, gave 0.172 g (20% yield) of the title compound: ¹H NMR (300 MHz, DMSO-d₆) δ 7.26-7.35 (m, 2H), 7.15-7.25 (m, 3H), 7.09 (d, 2H), 6.70-6.90 (m, 4H), 6.66 (d, 2H), 4.61 (s, 2H), 3.33 (s, 2H), 2.96 (d, 2H), 2.42 (d, 2H), 2.18 (s, 3H), 1.77-1.94 (m, 1H), 1.57-1.71 (m, 2H), 1.05-1.22 (m, 2H); MS (ESI) m/z 435.68 [M+H]⁺.

### Example 34

### 2-{5-[4-({(2E)-3-[4-(Dimethylamino)phenyl]prop-2-en-1-yl}amino)phenyl]-4-methyl-1,3-thiazol-2-yl}guanidine

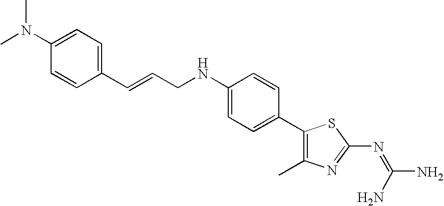

To a 16 mL screw top tube containing (E)-3-(4-dimethylaminophenyl)prop-2-enal (42 mg, 0.24 mmol) was added 2-[5-(4-aminophenyl)-4-methyl-1,3-thiazol-2-yl]guanidine (49 mg, 0.20 mmol) and sodium triacetoxyborohydride (424 mg, 2.00 mmol) followed by dichloromethane (1 mL). The resulting solution was shaken on a platform shaker (550 rpm) at room temperature. After 1 h a further addition of dichloromethane (3 mL) was made. After 22 h at room temperature the solvent was removed under reduced pressure, the residue dissolved in dimethyl sulfoxide, filtered and purified by preparative HPLC to afford 9.5 mg (11% yield) of the title compound: MS (ESI) m/z 407.48 [M+H]⁺.

### Example 35

### (2-Nitrophenyl)acetaldehyde

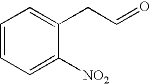

Dess-Martin periodinane (3.053 g, 7.2 mmol) was added in one portion to a stirred solution of 2-nitrophenyl alcohol (1.002 g, 6 mmol) in dichloromethane (50 mL). The mixture was stirred at room temperature under a nitrogen atmosphere for 1 h and was then poured into a 1:1 mixture of saturated aqueous sodium hydrogen carbonate and saturated aqueous sodium thiosulfate and the mixture was stirred vigorously for 30 min. The layers were separated and the aqueous layer was extracted with dichloromethane. The combined organic layers were dried over magnesium sulfate and concentrated in vacuo. Purification by column chromatography, using 0-50% ethyl acetate in hexanes as the eluent, gave 0.99 g (100% yield) of the title compound.

### Example 36

### Bromo(2-nitrophenyl)acetaldehyde

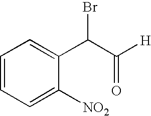

Bromine (0.339 mL, 6.6 mmol) was added dropwise over 10 min to a stirred solution of (2-nitrophenyl)acetaldehyde (0.990 g, 6 mmol) in dioxane (50 mL). The red solution was stirred for 30 min and then concentrated in vacuo. The crude product was used with no further purification.

### Example 37

### 1-[5-(2-Nitrophenyl)-1,3-thiazol-2-yl]guanidine

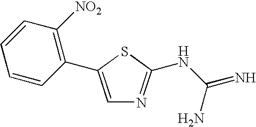

1-[Amino(imino)methyl]thiourea (0.726 g, 6.15 mmol) was added in one portion to a stirred solution of bromo(2-nitrophenyl)acetaldehyde (1.427, 6.15 mmol) in methanol (70 mL) and the mixture was stirred at 50° C. for 16 h. The reaction mixture was cooled to room temperature and concentrated to 5 mL. Diethyl ether was added and the mixture was concentrated in vacuo, 7 M ammonia in methanol was added and the solution was stirred for 15 min. The solution was concentrated and purified by column chromatography, using 0-10% 7 M ammonia in methanol and dichloromethane as the eluent, to give 0.43 g (27% yield) of the title compound: MS (ESI) m/z 264 [M+H]⁺.

### Example 38

### 1-[4-(4-Pyrrolidin-1-ylphenyl)-1,3-thiazol-2-yl]guanidine

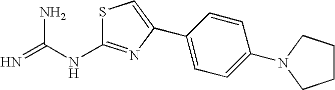

The title compound was synthesized as described for 1-[5-(2-nitrophenyl)-1,3-thiazol-2-yl]guanidine in 47% yield, starting from 2-bromo-1-(4-pyrrolidin-1-ylphenyl)ethanone and using ethanol as the solvent: MS (ESI) m/z 288 [M+H]⁺

### Example 39

### 1-{2-[(4-Aminophenyl)amino]-4′-methyl-4,5′-bi-1,3-thiazol-2′-yl}guanidine

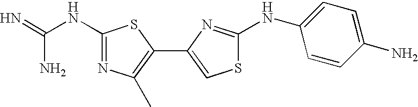

Ethanol (2 mL) was added to (4-amino-phenyl)-thiourea (35 mg, 0.21 mmol) and 1-[5-(bromoacetyl)-4-methyl-1,3-thiazol-2-yl]guanidine (55.4 mg, 0.2 mmol). The reaction mixture was stirred and heated at reflux for 1 h and then cooled. The resulting solid was recovered by filtration and purified by preparative HPLC to give 48.6 mg (70% yield) of the title compound: MS (ESI) m/z 346 [M+H]⁺

### Example 40

### 1-{4′-Methyl-2-[(4-phenoxyphenyl)amino]-4,5′-bi-1,3-thiazol-2′-yl}guanidine

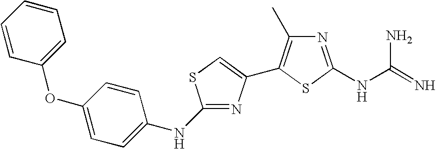

The title compound was synthesized as described for 1-{2-[(4-aminophenyl)amino]-4′-methyl-4,5′-bi-1,3-thiazol-2′-yl}guanidine in 27% yield, starting from 1-(4-phenoxyphenyl)-2-thiourea: MS (ESI) m/z 423 [M+H]⁺

### Example 41

### 1-{2-[(4-Methoxyphenyl)amino]-4′-methyl-4,5′-bi-1,3-thiazol-2′-yl}guanidine

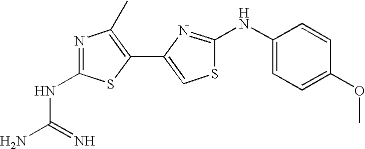

The title compound was synthesized as described for 1-{2-[(4-aminophenyl)amino]-4′-methyl-4,5′-bi-1,3-thiazol-2′-yl}guanidine in 55% yield, starting from 1-(4-methoxyphenyl)-2-thiourea: MS (ESI) m/z 361 [M+H]⁺

### Example 42

### 1-{4′-Methyl-2-[(4-nitrophenyl)amino]-4,5′-bi-1,3-thiazol-2′-yl}guanidine

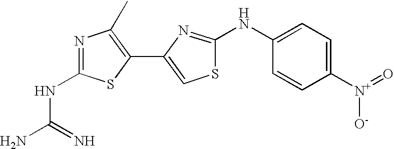

The title compound was synthesized as described for 1-{2-[(4-aminophenyl)amino]-4′-methyl-4,5′-bi-1,3-thiazol-2′-yl}guanidine in 87% yield, starting from 1-(4-nitrophenyl)-2-thiourea: MS (ESI) m/z 376 [M+H]⁺

### Example 43

### 1-[4′-Methyl-2-(pyridin-4-ylamino)-4,5′-bi-1,3-thiazol-2′-yl]guanidine

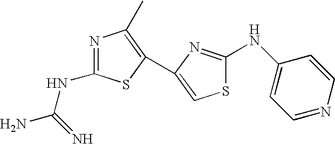

The title compound was synthesized as described for 1-{2-[(4-aminophenyl)amino]-4′-methyl-4,5′-bi-1,3-thiazol-2′-yl}guanidine in 91% yield, starting from N-(4-pyridyl)thiourea: MS (ESI) m/z 332 [M+H]⁺

### Example 44

### 1-[2-(Dimethylamino)-4′-methyl-4,5′-bi-1,3-thiazol-2′-yl]guanidine

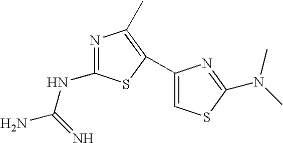

The title compound was synthesized as described for 1-{2-[(4-aminophenyl)amino]-4′-methyl-4,5′-bi-1,3-thiazol-2′-yl}guanidine in 76% yield, starting from 1,1-dimethyl-2-thiourea: MS (ESI) m/z 283 [M+H]⁺

### Example 45

### 1-[2-(Allylamino)-4′-methyl-4,5′-bi-1,3-thiazol-2′-yl]guanidine

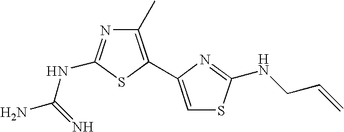

The title compound was synthesized as described for 1-{2-[(4-aminophenyl)amino]-4′-methyl-4,5′-bi-1,3-thiazol-2′-yl}guanidine in 39% yield, starting from allylthiourea: MS (ESI) m/z 295 [M+H]⁺

### Example 46

### 1-[2-(2,6-Dichloropyridin-4-yl)-4′-methyl-4,5′-bi-1,3-thiazol-2′-yl]guanidine

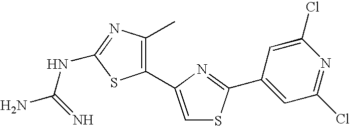

The title compound was synthesized as described for 1-{2-[(4-aminophenyl)amino]-4′-methyl-4,5′-bi-1,3-thiazol-2′-yl}guanidine in 73% yield, starting from 2,6-dichloropyridine-4-thiocarboxamide: MS (ESI) m/z 385 [M+H]⁺.

### Example 47

### Methyl 4-{[methyl(phenyl)amino]methyl}benzoate

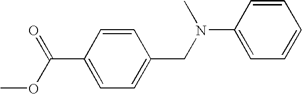

N-methyl aniline (1.18 g, 11.0 mmol) was added in a single portion to a stirred solution of methyl 4-(bromomethyl)benzoate (2.29 g, 10.0 mmol) in anhydrous N,N-dimethylacetamide (50 mL). Calcium carbonate (2.00 g, 20.0 mmol) was added in a single portion and the reaction was heated at 60° C. for 16 h. The mixture was then poured into water (100 mL) and the resulting suspension was extracted with ethyl acetate (3×50 mL). The combined organics were washed with brine, dried over magnesium sulfate, filtered and concentrated to dryness. Purification by column chromatography, using isohexane/ethyl acetate (5:1) afforded 2.10 g (82% yield) of the title compound: MS (ESI) m/z 256.02 [M+H]⁺.

### Example 48

### 4-{[Methyl(phenyl)amino]methyl}benzoic acid

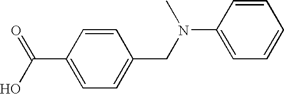

A 1 M aqueous solution of sodium hydroxide (16.0 mL, 16.0 mmol) was added to a stirred solution of methyl 4-{[methyl(phenyl)amino]methyl}benzoate (2.0 g, 8.0 mmol) in methanol (25 mL). The reaction was stirred for 16 h at room temperature and then evaporated to dryness. The resulting residue was dissolved in water (25 mL) and this solution was acidified with 2 M aqueous hydrochloric acid. This was extracted with ethyl acetate (2×25 mL) and the combined organics were dried over magnesium sulfate and concentrated to dryness to afford 1.52 g (79% yield) of the title compound: MS (ESI) m/z 240.02 [M−H]⁺.

### Example 49

### N-[4-(2-{[Amino(imino)methyl]amino}-4-methyl-1,3-thiazol-5-yl)phenyl]-5-fluoro-1H-indole-2-carboxamide

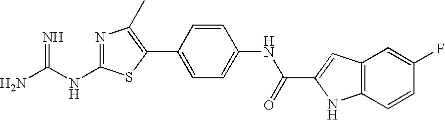

A stirred solution of O-(7-azabenzotriazol-1-yl)-N,N,N′,N′-tetramethyluronium hexafluorophosphate (105 mg, 0.28 mmol) in anhydrous N,N-dimethylformamide (2 mL) was added to a reaction tube containing 5-fluoro-1H-indole-2-carboxylic acid (45 mg, 0.25 mmol). N,N-Diisopropylethylamine (0.34 mL, 2.00 mmol) was added and the mixture was stirred for 30 min. A stirred solution of 2-[5-(4-aminophenyl)-4-methyl-1,3-thiazol-2-yl]guanidine (62 mg, 0.25 mmol, described in Alig L.; Edenhofer A.; Hilpert K.; Weller T. Preparation of thiazolecarboxamides as vitronectin receptor antagonists. EP 928790 A1, 19990714) in anhydrous N,N-dimethylformamide (2 mL) was then added in a single portion and the reaction was stirred for 16 h at room temperature. The reaction mixture was then purified directly by reverse phase HPLC to afford 40.1 mg, (39% yield) of the title compound: MS (ESI) m/z 409.88 [M+H]⁺.

### Example 50

### N-[4-(2-{[amino(imino)methyl]amino}-4-methyl-1,3-thiazol-5-yl)phenyl]-4-{[methyl(phenyl)amino]methyl}benzamide

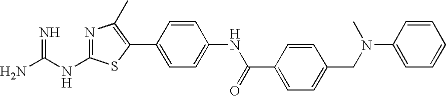

A stirred solution of O-(7-azabenzotriazol-1-yl)-N,N,N′,N′-tetramethyluronium hexafluorophosphate (105 mg, 0.28 mmol) in anhydrous N,N-dimethylformamide (2 mL) was added to a reaction tube containing 4-{[methyl(phenyl)amino]methyl}benzoic acid (60 mg, 0.25 mmol). N,N-Diisopropylethylamine (0.34 mL, 2.00 mmol) was then added in a single portion and the mixture was stirred for 30 min. A stirred solution of 2-[5-(4-aminophenyl)-4-methyl-1,3-thiazol-2-yl]guanidine (62 mg, 0.25 mmol described in Alig L.; Edenhofer A.; Hilpert K.; Weller T. Preparation of thiazolecarboxamides as vitronectin receptor antagonists. EP 928790 A1, 19990714) in anhydrous N,N-dimethylformamide (2 mL) was then added in a single portion and the reaction was stirred for 16 h at room temperature. The reaction mixture was then purified directly by reverse phase HPLC to afford 59.0 mg, (50% yield) of the title compound: MS (ESI) m/z 469.95 [M−H]⁺.

### Example 51

### 2-{[Amino(imino)methyl]amino}-N-(9-oxo-9H-fluoren-2-yl)-1,3-thiazole-4-carboxamide

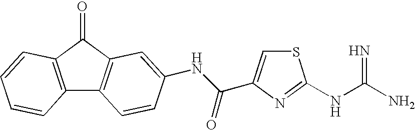

To a solution of 2-amino-9H-fluoren-9-one (19.5 mg, 0.1 mmol) in N,N-dimethylformaide (1 mL) was added a solution of 24{[(tert-butoxycarbonyl)amino](imino)methyl}amino)-1,3-thiazole-4-carboxylic acid (28.6 mg, 0.1 mmol), O-benzotriazol-1-yl-N,N,N′,N′-tetramethyluronium tetrafluoroborate (38.5 mg, 0.12 mmol) and N,N-diisopropylethylamine (69.8 μL, 0.4 mmol) in N,N-dimethylformamide (1 mL). The vial was capped and shaken for 16 h at room temperature. The reaction mixture was evaporated using a vacuum centrifuge. To the dry crude product was added trifluoroacetic acid (400 μL), and the reaction mixture was shaken for 2 h. The mixture was evaporated to dryness using a vacuum centrifuge. Purification by preparative HPLC gave 19.2 mg (52.8% yield) of the title compound: MS (ESI) m/z 364 [M+H]⁺.

### Example 52

### 2-{[Amino(imino)methyl]amino}-N-1H-indol-5-yl-1,3-thiazole-4-carboxamide

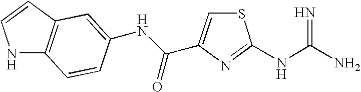

The title compound was synthesized as described for 2-{[amino(imino)methyl]amino}-N-(9-oxo-9H-fluoren-2-yl)-1,3-thiazole-4-carboxamide in 32% yield, starting from 1H-indol-5-amine: MS (ESI) m/z 301 [M+H]⁺.

### Example 53

### 1-(2′-Amino-4′-methyl-4,5′-bi-1,3-thiazol-2-yl)guanidine dihydrobromide

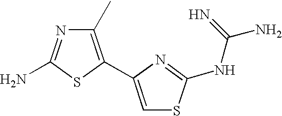

1-(2-Amino-4-methyl-1,3-thiazol-5-yl)-2-bromoethanone hydrobromide (1.8 g, 5.7 mmol, described in Quattropani A.; Dorbais J.; Covini D.; Desforges G.; Rueckle T. Preparation of 4,5′-bithiazole and 4-(oxazol-5-yl)thiazole derivatives as phosphoinositide-3 kinase inhibitors with therapeutic uses. WO 2006125805 A1 20061130) was dissolved in boiling ethanol (70 mL), treated with a hot ethanolic solution of 1-[amino(imino)methyl]thiourea (710 mg, 6.0 mmol) and the resulting mixture was heated at reflux for 1 h. A small sample was taken out, evaporated and treated with methanol/acetone to induce solidification. The remaining reaction mixture was concentrated to approximately 15 mL, seeded with the solid obtained previously, stored over the weekend, filtered and dried to give 1 g (69% yield) of the title compound: Elemental Analysis; Found C, 23.5; H, 3.1; N, 19.9; C₈H₁₀N₆S₂×2 HBr requires C, 23.1; H, 2.91; N, 20.2%

### Example 54

### 3,5-Diamino-6-chloropyrazine-2-carbothioamide

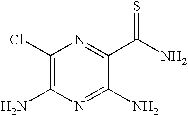

Diethylamine (126.34 g, 1.727 mol) was added to a solution of 3,5-diamino-6-chloropyrazine-2-carbonitrile (116.5 g, 0.687 mol, described in Cragoe E.; Jones J. 2,4-Diamino-6-haloptheridines and processes for their preparation. U.S. Pat. No. 3,487,082, 19691230) in anhydrous N,N-dimethylformamide (3000 mL) and the resulting mixture was heated to 50° C. Hydrogen sulfide (g) was bubbled through the reaction mixture for 4 h keeping the temperature between 50-57° C. The addition of hydrogen sulfide was stopped and the mixture was stirred for another 30 min. The reaction mixture was flushed with nitrogen for 7 h, cooled to 20° C. and left for 1 h. The formed solid was removed by filtration, washed with water and dried in a vacuum oven at 60° C. for 8 h to give 101.7 g (72.7% yield) of the title compound.

### Example 55

### 1-[2-(3,5-Diamino-6-chloropyrazin-2-yl)-4′-methyl-4,5′-bi-1,3-thiazol-2′-yl]guanidine

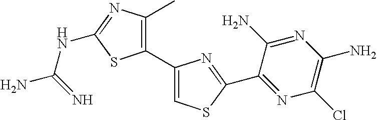

The title compound was synthesized as described for 1-{2-[(4-aminophenyl)amino]-4′-methyl-4,5′-bi-1,3-thiazol-2′-yl}guanidine in 89% yield, starting from 3,5-diamino-6-chloro-pyrazine-2-carbothioic acid amide.: MS (ESI) m/z 382 [M+H]⁺

### Assays

Compounds were tested in at least one of the following assays:

### β-Secretase Enzyme

The enzyme used in the IGEN Cleavage-, Fluorescent-, TR-FRET- and the BiaCore assay is described as follows:

The soluble part of the human β-Secretase (AA 1-AA 460) was cloned into the ASP2-Fc10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

### IGEN Cleavage Assay

Enzyme is diluted 1:30 in 40 mM MES pH 5.0. Stock substrate is diluted to 12 μM in 40 mM MES pH 5.0. Compounds are diluted to the desired concentration in dimethylsulphoxide (final dimethylsulphoxide concentration in assay is 5%). The assay is done in a 96 well PCR plate from Greiner (#650201). Compound in dimethylsulphoxide (3 μL) is added to the plate, and then enzyme is added (27 μL) and pre-incubated with compound for 10 minutes. The reaction is started with substrate (30 μL). The final dilution of enzyme is 1:60 and the final concentration of substrate is 6 μM. After a 20 minute reaction at room temperature, the reaction is stopped by removing 10 μl of the reaction mix and diluting it 1:25 in 0.2 M Trizma-HCl, pH 8.0. Compounds are diluted and added to the plate by the Biomek FX or by hand, then all the rest of the liquid handling is done with on the Biomek 2000 instrument.

All antibodies and the streptavidin coated beads are diluted in PBS containing 0.5% BSA and 0.5% Tween20. The product is quantified by adding 50 μl of a 1:5000 dilution of the neoepitope antibody to 50 μl of the 1:25 dilution of the reaction mix. Then, 100 μl of PBS (0.5% BSA, 0.5% Tween20) containing 0.2 mg/mL IGEN beads (Dynabeads M-280) and a 1:5000 dilution of ruthinylated goat anti-rabbit (Ru-GαR) antibody is added. The final dilution of neoepitope antibody is 1:20,000, the final dilution of Ru-GAR is 1:10,000 and the final concentration of beads is 0.1 mg/mL. The mixture is read on the IGEN instrument (BioVeris) with the Abbiochemial assay program after a 2-hour incubation with shaking at room temperature.

### Fluorescent Assay

Enzyme is diluted 1:25 in 40 mM MES pH 5.0. Stock substrate (Dabcyl) is diluted to 30 μM in 40 mM MES pH 5.0. Enzyme and substrate stock solutions are kept on ice until placed in the stock plates. The Biomek FX instrument is used to do all liquid handling. Enzyme (9 μL) together with 1 μl of compound in dimethylsulphoxide is added to the plate and pre-incubated for 10 minutes. When a dose response curve is being tested for a compound, the dilutions are done in neat dimethylsulphoxide. Substrate (10 μl) is added and the reaction proceeds in the dark for 25 minutes at room temperature. The assay is done in a Corning 384 well round bottom, low volume, non-binding surface (Corning #3676). The final dilution of enzyme is 1:50, and the final concentration of substrate is 15 μM (Km of 25 μM). The fluorescence of the product is measured on a Victor II plate reader with an excitation wavelength of 360 nm and an emission wavelength of 485 nm using the protocol for labelled Edans peptide. The dimethylsulphoxide control defines 100% activity level and 0% activity is defined by exclusion of the enzyme (using 40 mM MES pH 5.0 buffer instead).

### TR-FRET Assay

Dilute the enzyme (truncated form) to 6 μg/mL (stock 1.3 mg/mL) and the substrate (Europium)CEVNLDAEFK(Qsy7) to 200 nM (stock 60 μM) in reaction buffer (NaAcetate, chaps, triton x-100, EDTA pH4.5). The Biomek FX is used for all liquid handling and the enzyme and substrate solutions are kept on ice until they are placed in Biomek FX. Enzyme (9 μl) is added to the plate then 1 μl of compound in dimethylsulphoxide is added, mixed and pre-incubated for 10 minutes. Substrate (10 μl) is then added, mixed and the reaction proceeds in the dark for 15 minutes at room temperature. The reaction is stopped with the addition of Stop solution (7 NaAcetate pH 9). The fluorescence of the product is measured on a Victor II plate reader with an excitation wavelength of 340 nm and an emission wavelength of 615 nm. The assay is done in a Costar 384 well round bottom, low volume, non-binding surface (Corning #3676). The final concentration of the enzyme is 0.3 nM; the final concentration of substrate is 100 nM (Km of ˜250 nM). The dimethylsulphoxide control defines the 100% activity level and 0% activity is defined by only addition of the peptide substrate. A control inhibitor is also used in dose response assays and has an IC50 of 575 nM.

### Beta-Secretase Whole Cell Assay

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

### Cell Culture

HEK293 cells stably expressing human wild-type APP (HEK293-APP695) were grown at 37° C. in DMEM containing 4500 g/L glucose, GlutaMAX and sodium pyruvate supplemented with 10% FBS, 1% non-essential amino acids and 0.1 mg/mL of the selection antibiotic zeocin.

### A/β40 Release Assay

Cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) and containing test compounds in a final dimethylsulphoxide concentration of 1%. Cells were exposure to test compounds for 24 h at 37° C., 5% CO₂. To quantify the amount of released Aβ, 100 μL cell medium was transferred to a round bottom polypropylene 96-well plate (assay plate). The cell plate was saved for ATP assay as described in ATP assay below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 mg/mL of a ruthenylated goat anti-rabbit antibody and 0.2 mg/mL of streptavidin coated Dynabeads was added per well. The plate was vigorously shaken at room temperature for 1-2 h. The plate was then measured for electro-chemiluminescence counts in an IGEN M8 Analyzer. An Aβ standard curve was obtained using standards at concentrations 20, 10, 2 and 0.2 ng Aβ/mL in the cell culture medium with penicillin and streptomycin (100 U/mL, 100 μg/mL, respectively).

### ATP Assay

As indicated above, after transferring 100 μL medium from the cell plate for Aβ40 detection, the plate was used to analyse cytotoxicity using the ViaLight™ Plus cell proliferation/cytotoxicity kit from Cambrex BioScience that measures total cellular ATP. The assay was performed according to the manufacture's protocol. Briefly, 50 μl cell lysis reagent was added per well. The plates were incubated at room temperature for 10 min. Two min after addition of 100 μL reconstituted ViaLight™ Plus ATP reagent, the luminescence was measured in a Wallac Victor² 1420 multilabel counter.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/mL in 20 mM Na Acetate pH 4.5, and then the solutions were centrifuged at 14K rpm to remove any particulates. Carboxyl groups on the dextran layer were activated by injecting a one to one is mixture of 0.5 M N-ethyl-N′ (3-dimethylaminopropyl)-carbodiimide (EDC) and 0.5 M N-hydroxysuccinimide (NHS) at 5 μL/min for 7 min. Then the stock solution of the control peptide was injected in channel 1 for 7 min at 5 μL/min., and then the remaining activated carboxyl groups were blocked by injecting 1 M ethanolamine for 7 min at 5 μL/min.

### Assay Protocol

The BACE Biacore assay was done by diluting BACE to 0.5 μM in Na Acetate buffer at pH 4.5 (running buffer minus dimethylsulphoxide). The diluted BACE was mixed with dimethylsulphoxide or compound diluted in dimethylsulphoxide at a final concentration of 5% dimethylsulphoxide. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethylsulphoxide control was defined as 100% and the effect of the compound was reported as percent inhibition of the dimethylsulphoxide control.

### Results

Typical Kᵢ values for the compounds of the present invention are in the range of about 1 to about 10,000 nM. Biological data on an example is given below in Table 2.

TABLE 2

| Example No. | IC50 in TR-FRET Assay |
|---|---|
| 17 | 4.47 μM |
| 18 | 6.03 μM |
| 20 | 5.01 μM |

1. A compound of formula I: 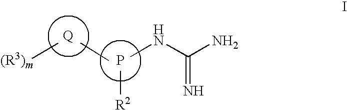
wherein
P is thiazole;
Q is independently selected from phenyl, thiazole, C₀₋₃alkylCONR⁴R⁵, C₀₋₃alkylNR⁴COR⁵, C₀₋₃alkylNR⁴(SO₂)R⁵, and C₀₋₃alkyl(SO₂)NR⁴R⁵;
R² is independently selected from hydrogen, halogen, C₁₋₆alkyl, CN, C₀₋₆alkylOR⁴, fluoromethyl, difluoromethyl, trifluoromethyl, fluoromethoxy, difluoromethoxy, trifluoromethoxy, C₂₋₆alkenyl and C₂₋₆alkynyl;
R³ is independently selected from halogen, nitro, CHO, CN, OC₁₋₆alkylCN, OR⁴, OC₁₋₆alkylOR⁴, fluoromethyl, difluoromethyl, trifluoromethyl, fluoromethoxy, difluoromethoxy, trifluoromethoxy, NR⁴R⁵, OC₁₋₆alkylNR⁴R⁵, NR⁴COR⁵, CO₂R⁴, CONR⁴R⁵, OC₁₋₆alkylCONR⁴R⁵, OC₁₋₆alkylNR⁴(CO)R⁵, NR⁴(CO)R⁵, O(CO)NR⁴R⁵, NR⁴(CO)OR⁵, NR⁴(CO)NR⁴R⁵, O(CO)R⁴, COR⁴, OC₁₋₆alkylCOR⁴, SR⁴, (SO₂)NR⁴R⁵, OC₁₋₆alkylNR⁴(SO₂)R⁵, OC₀₋₆alkyl(SO₂)NR⁴R⁵, (SO)NR⁴R⁵, OC₁₋₆alkyl(SO)NR⁴R⁵, SO₃R⁴, NR⁴(SO)R⁵, NR⁴(SO₂)R⁵, OC₁₋₆alkylNR⁴(SO)R⁵, OC₀₋₆alkylSO₂R⁴, SO₂R⁴, SOR⁴, C₁₋₆alkyl, C₂₋₆alkenyl, C₂₋₆alkynyl, C₃₋₆cycloalkyl, aryl and heteroaryl, wherein said C₁₋₆alkyl, C₂₋₆alkenyl, C₂₋₆alkynyl, C₃₋₆cycloalkyl, aryl or heteroaryl may be optionally substituted with one or more A;
R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylC₃₋₆heterocyclyl, C₀₋₆alkylaryl, C₀₋₆alkylheteroaryl and C₁₋₆alkylNR⁶R⁷, wherein said C₁₋₆alkyl, C₂₋₆alkenyl, C₀₋₆alkylC₃₋₆heterocyclyl, C₀₋₆alkylaryl or C₀₋₆alkylheteroaryl may be optionally substituted by one or more A; or
R⁴ and R⁵ may together form a 5 or 6 membered heterocyclic ring containing one or more heteroatoms selected from N, O or S, which heterocyclic ring may be optionally substituted by one or more A;
A is independently selected from oxo, halogen, nitro, CN, OR⁶, C₁₋₆alkyl, C₀₋₆alkylaryl, heteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, NR⁶R⁷, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₀₋₆alkylaryl, heteroaryl, C₀₋₆alkylheterocyclyl or C₀₋₆alkylC₃₋₆cycloalkyl may be optionally substituted with one or more substituents independently selected from halogen, nitro, cyano, OR⁶, C₁₋₆alkyl, fluoromethyl, difluoromethyl, trifluoromethyl, fluoromethoxy, difluoromethoxy, trifluoromethoxy, and NR⁶R⁷;
R⁶ and R⁷ are independently selected from hydrogen, C₁₋₆alkyl, C₀₋₆alkylaryl, fluoromethyl, difluoromethyl and trifluoromethyl, or
R⁶ and R⁷ may together form an optionally substituted 5, 6 or 7 membered heterocyclic ring containing one or more heteroatoms selected from N, O or S;
m is 0, 1, 2 or 3;
provided that when Q is C₀₋₃alkylCONR⁴R⁵ and R⁴ or R⁵ is C₀₋₆alkylC₃₋₆heterocyclyl, said C₀₋₆alkylC₃₋₆heterocyclyl is not benzotriazole; and
provided that when Q is C₀₋₃alkylCONR⁴R⁵, C₀₋₃alkylNR⁴COR⁵, C₀₋₃alkylNR⁴(SO₂)R⁵ or C₀₋₃alkyl(SO₂)NR⁴R⁵, m is 0; and
provided that the following compounds are excluded:
1-[2′-(Aminomethyl)-4,4′-bi-1,3-thiazol-2-yl]guanidine;
N-[(2′-{[Amino(imino)methyl]amino}-4,4′-bi-1,3-thiazol-2-yl)methyl]acetamide;
N-[(2-{[Amino(imino)methyl]amino}-4,5′-bi-1,3-thiazol-2′-yl)methyl]acetamide;
1-[2′-(Aminomethyl)-4,5′-bi-1,3-thiazol-2-yl]guanidine;
2′-{[Amino(imino)methyl]amino}-4,4′-bi-1,3-thiazole-2-carboxamide;
1-{2′-[(Dimethylamino)methyl]-4,4′-bi-1,3-thiazol-2-yl}guanidine;
1-(2′-Cyano-4,4′-bi-1,3-thiazol-2-yl)guanidine;
1-[2′-(Cyanomethyl)-4,4′-bi-1,3-thiazol-2-yl]guanidine;
N-(2′-{[Amino(imino)methyl]amino}-4,4′-bi-1,3-thiazol-2-yl)acetamide;
Ethyl 2′-{[amino(imino)methyl]amino}-4,4′-bi-1,3-thiazole-2-carboxylate;
1-[2′-(2-Aminoethyl)-4,4′-bi-1,3-thiazol-2-yl]guanidine;
N-[2-(2′-{[Amino(imino)methyl]amino}-4,4′-bi-1,3-thiazol-2-yl)ethyl]acetamide;
1-[4′-Methyl-2′-(methylamino)-4,5′-bi-1,3-thiazol-2-yl]guanidine;
1-{2′-[Formyl(methyl)amino]-4′-methyl-4,5′-bi-1,3-thiazol-2-yl}guanidine;
1-(2′-Amino-4,4′-bi-1,3-thiazol-2-yl)guanidine;
1-(4,4′-Bi-1,3-thiazol-2-yl)guanidine;
1-[2′-(Methylamino)-4,4′-bi-1,3-thiazol-2-yl]guanidine;
1-(4-Phenyl-1,3-thiazol-2-yl)guanidine;
1-[4-(3-Aminophenyl)-1,3-thiazol-2-yl]guanidine;
N-[3-(2-{[Amino(imino)methyl]amino}-1,3-thiazol-4-yl)phenyl]acetamide;
1-{4-[3-(2-Oxopropyl)phenyl]-1,3-thiazol-2-yl}guanidine;
N-[3-(2-{[Amino(imino)methyl]amino}-1,3-thiazol-5-yl)phenyl]acetamide;
1-[4-(4-tert-Butylphenyl)-1,3-thiazol-2-yl]guanidine;
1-[4-(4-Nitrophenyl)-1,3-thiazol-2-yl]guanidine;
1-[4-(4-Fluorophenyl)-1,3-thiazol-2-yl]guanidine;
1-[4-(4-Hydroxyphenyl)-1,3-thiazol-2-yl]guanidine;
1-[4-(4-Aminophenyl)-1,3-thiazol-2-yl]guanidine;
1-[4-(4-Methoxyphenyl)-1,3-thiazol-2-yl]guanidine;
1-[4-(2-Chlorophenyl)-1,3-thiazol-2-yl]guanidine;
1-[4-(3-Chlorophenyl)-1,3-thiazol-2-yl]guanidine;
1-[4-(2-Methylphenyl)-1,3-thiazol-2-yl]guanidine;
1-[4-(3-Methylphenyl)-1,3-thiazol-2-yl]guanidine;
1-(4-{3-[(Dimethylamino)methyl]phenyl}-1,3-thiazol-2-yl)guanidine;
1-[4-(2-Methoxyphenyl)-1,3-thiazol-2-yl]guanidine;
1-[4-(3-Hydroxyphenyl)-1,3-thiazol-2-yl]guanidine;
1-[4-(3,4-Dihydroxyphenyl)-1,3-thiazol-2-yl]guanidine;
1-[4-(3,4-Dihydroxyphenyl)-5-methyl-1,3-thiazol-2-yl]guanidine;
1-[4-(4-Isopropylphenyl)-1,3-thiazol-2-yl]guanidine;
1-[4-(3-Methoxyphenyl)-1,3-thiazol-2-yl]guanidine;
N-[3-(2-{[Amino(imino)methyl]amino}-1,3-thiazol-4-yl)benzyl]acetamide;
N-(3-{[4-(2-{[Amino(imino)methyl]amino}-1,3-thiazol-4-yl)phenyl]amino}-3-oxopropyl)-N-benzylbenzamide;
1-[4-(4-Chlorophenyl)-1,3-thiazol-2-yl]guanidine;
1-[4-(4-Methylphenyl)-1,3-thiazol-2-yl]guanidine;
1-{4-[3-(Dimethylamino)phenyl]-1,3-thiazol-2-yl}guanidine;
1-(4-{2-[(Dimethylamino)methyl]phenyl}-1,3-thiazol-2-yl)guanidine;
1-(4-Biphenyl-4-yl-1,3-thiazol-2-yl)guanidine;
1-{4-[4-(Dimethylamino)phenyl]-1,3-thiazol-2-yl}guanidine;
1-[4-(3,4-Dimethoxyphenyl)-1,3-thiazol-2-yl]guanidine;
1-[4-(2-Hydroxyphenyl)-1,3-thiazol-2-yl]guanidine;
6-{[3-(2-{[Amino(imino)methyl]amino}-4-methyl-1,3-thiazol-5-yl)phenyl]amino}-6-oxohexanoic acid;
6-{[4-(2-{[Amino(imino)methyl]amino}-4-methyl-1,3-thiazol-5-yl)phenyl]amino}-6-oxohexanoic acid;
4-{[4-(2-{[Amino(imino)methyl]amino}-4-methyl-1,3-thiazol-5-yl)benzoyl]amino}-3-(4-chlorophenyl)butanoic acid;
4-{[3-(2-{[Amino(imino)methyl]amino}-4-methyl-1,3-thiazol-5-yl)benzoyl]amino}-3-(4-chlorophenyl)butanoic acid;
3-{[4-(2-{[Amino(imino)methyl]amino}-4-methyl-1,3-thiazol-5-yl)benzoyl]amino}-3-phenylpropanoic acid;
3-{[3-(2-{[Amino(imino)methyl]amino}-4-methyl-1,3-thiazol-5-yl)benzoyl]amino}-3-phenylpropanoic acid;
4-{[3-(2-{[Amino(imino)methyl]amino}-1,3-thiazol-4-yl)benzoyl]amino}-3-(4-chlorophenyl)butanoic acid;
5-{[3-(2-{[Amino(imino)methyl]amino}-4-methyl-1,3-thiazol-5-yl)phenyl]amino}-3-(4-methoxyphenyl)-5-oxopentanoic acid;
5-{[3-(2-{[Amino(imino)methyl]amino}-4-methyl-1,3-thiazol-5-yl)phenyl]amino}-5-oxo-3-pyridin-3-ylpentanoic acid;
5-{[3-(2-{[Amino(imino)methyl]amino}-4-ethyl-1,3-thiazol-5-yl)phenyl]amino}-5-oxo-3-phenylpentanoic acid;
5-{[3-(2-{[Amino(imino)methyl]amino}-4-propyl-1,3-thiazol-5-yl)phenyl]amino}-5-oxo-3-phenylpentanoic acid;
5-{[3-(2-{[Amino(imino)methyl]amino}-4-butyl-1,3-thiazol-5-yl)phenyl]amino}-5-oxo-3-phenylpentanoic acid;
5-{[3-(2-{[Amino(imino)methyl]amino}-4-pentyl-1,3-thiazol-5-yl)phenyl]amino}-5-oxo-3-phenylpentanoic acid;
5-{[3-(2-{[Amino(imino)methyl]amino}-4-methyl-1,3-thiazol-5-yl)phenyl]amino}-3-(4-chlorophenyl)-5-oxopentanoic acid;
5-{[3-(2-{[Amino(imino)methyl]amino}-4-methyl-1,3-thiazol-5-yl)phenyl]amino}-3-hydroxy-5-oxopentanoic acid;
5-{[3-(2-{[Amino(imino)methyl]amino}-4-methyl-1,3-thiazol-5-yl)phenyl]amino}-5-oxopentanoic acid;
5-{[3-(2-{[Amino(imino)methyl]amino}-4-methyl-1,3-thiazol-5-yl)phenyl]amino}-3-methyl-5-oxopentanoic acid;
5-{[3-(2-{[Amino(imino)methyl]amino}-4-methyl-1,3-thiazol-5-yl)phenyl]amino}-3-(3-bromophenyl)-5-oxopentanoic acid:
5-{[3-(2-{[Amino(imino)methyl]amino}-4-methyl-1,3-thiazol-5-yl)phenyl]amino}-3-(3,5-dichloro-2-hydroxyphenyl)-5-oxopentanoic acid;
5-{[3-(2-{[Amino(imino)methyl]amino}-4-methyl-1,3-thiazol-5-yl)phenyl]amino}-3-(3-methoxyphenyl)-5-oxopentanoic acid;
5-{[3-(2-{[Amino(imino)methyl]amino}-4-methyl-1,3-thiazol-5-yl)-4-chlorophenyl]amino}-5-oxo-3-phenylpentanoic acid;
5-{[5-(2-{[Amino(imino)methyl]amino}-4-methyl-1,3-thiazol-5-yl)-2-chlorophenyl]amino}-5-oxo-3-phenylpentanoic acid;
4-{[3-(2-{[Amino(imino)methyl]amino}-4-methyl-1,3-thiazol-5-yl)benzoyl]amino}-2-[(butylsulfonyl)amino]butanoic acid;
3-({[3-(2-{[Amino(imino)methyl]amino}-4-methyl-1,3-thiazol-5-yl)phenyl]carbamoyl}amino)-3-phenylpropanoic acid;
5-{[3-(2-{[Amino(imino)methyl]amino}-4-methyl-1,3-thiazol-5-yl)phenyl](methyl)amino}-5-oxo-3-phenylpentanoic acid;
4-{[4-(2-{[Amino(imino)methyl]amino}-4-methyl-1,3-thiazol-5-yl)phenyl]amino}-4-oxobutanoic acid;
4-{[3-(2-{[Amino(imino)methyl]amino}-4-methyl-1,3-thiazol-5-yl)phenyl]amino}-4-oxobutanoic acid;
5-{[3-(2-{[Amino(imino)methyl]amino}-4-methyl-1,3-thiazol-5-yl)phenyl]amino}-5-oxo-3-phenylpentanoic acid;
5-{[4-(2-{[Amino(imino)methyl]amino}-4-methyl-1,3-thiazol-5-yl)phenyl]amino}-5-oxo-3-phenylpentanoic acid;
1-[4-Methyl-5-(4-nitrophenyl)-1,3-thiazol-2-yl]guanidine;
1-[4-Methyl-5-(3-nitrophenyl)-1,3-thiazol-2-yl]guanidine;
Methyl 4-(2-{[amino(imino)methyl]amino}-4-methyl-1,3-thiazol-5-yl)benzoate;
Methyl 3-(2-{[amino(imino)methyl]amino}-4-methyl-1,3-thiazol-5-yl)benzoate;
1-[5-(3-Aminophenyl)-4-methyl-1,3-thiazol-2-yl]guanidine;
1-[5-(4-Aminophenyl)-4-methyl-1,3-thiazol-2-yl]guanidine;
4-(2-{[Amino(imino)methyl]amino}-4-methyl-1,3-thiazol-5-yl)benzoic acid;
3-(2-{[Amino(imino)methyl]amino}-4-methyl-1,3-thiazol-5-yl)benzoic acid;
Methyl 6-{[4-(2-{[amino(imino)methyl]amino}-4-methyl-1,3-thiazol-5-yl)phenyl]amino}-6-oxohexanoate;
Methyl 6-{[3-(2-{[amino(imino)methyl]amino}-4-methyl-1,3-thiazol-5-yl)phenyl]amino}-6-oxohexanoate;
Ethyl 4-{[4-(2-{[amino(imino)methyl]amino}-4-methyl-1,3-thiazol-5-yl)benzoyl]amino}-3-(4-chlorophenyl)butanoate;
Ethyl 4-{[3-(2-{[amino(imino)methyl]amino}-4-methyl-1,3-thiazol-5-yl)benzoyl]amino}-3-(4-chlorophenyl)butanoate;
Ethyl 3-{[4-(2-{[amino(imino)methyl]amino}-4-methyl-1,3-thiazol-5-yl)benzoyl]amino}-3-phenylpropanoate;
Ethyl 3-{[3-(2-{[amino(imino)methyl]amino}-4-methyl-1,3-thiazol-5-yl)benzoyl]amino}-3-phenylpropanoate;
5-{[3-(2-{[Amino(imino)methyl]amino}-1,3-thiazol-4-yl)phenyl]amino}-5-oxo-3-phenylpentanoic acid;
1-[4-(3-Nitrophenyl)-1,3-thiazol-2-yl]guanidine;
3-(2-{[Amino(imino)methyl]amino}-1,3-thiazol-4-yl)benzoic acid;
1-[4-Ethyl-5-(3-nitrophenyl)-1,3-thiazol-2-yl]guanidine;
1-[5-(3-Aminophenyl)-4-ethyl-1,3-thiazol-2-yl]guanidine;
1-[5-(3-Aminophenyl)-4-propyl-1,3-thiazol-2-yl]guanidine;
1-[5-(3-Nitrophenyl)-4-propyl-1,3-thiazol-2-yl]guanidine;
1-[4-Butyl-5-(3-nitrophenyl)-1,3-thiazol-2-yl]guanidine;
1-[5-(3-Aminophenyl)-4-butyl-1,3-thiazol-2-yl]guanidine;
1-[5-(3-Aminophenyl)-4-pentyl-1,3-thiazol-2-yl]guanidine;
1-[5-(3-Nitrophenyl)-4-pentyl-1,3-thiazol-2-yl]guanidine;
1-[5-(2-Chloro-5-nitrophenyl)-4-methyl-1,3-thiazol-2-yl]guanidine;
1-[5-(4-Chloro-3-nitrophenyl)-4-methyl-1,3-thiazol-2-yl]guanidine;
Ethyl 3-({[3-(2-{[amino(imino)methyl]amino}-4-methyl-1,3-thiazol-5-yl)phenyl]carbamoyl}amino)-3-phenylpropanoate;
Ethyl 5-{[3-(2-{[amino(imino)methyl]amino}-4-methyl-1,3-thiazol-5-yl)phenyl]amino}-5-oxo-3-phenylpentanoate;
1-[5-(3-{[(2,5-Dioxopyrrolidin-1-yl)methyl]amino}phenyl)-4-methyl-1,3-thiazol-2-yl]guanidine;
1-{4-Methyl-5-[3-(methylamino)phenyl]-1,3-thiazol-2-yl}guanidine;
1-[4-(3-{4-[Methyl(phenyl)amino]butoxy}phenyl)-1,3-thiazol-2-yl]guanidine;
1-{4-[3-(4-Chlorobutoxy)phenyl]-1,3-thiazol-2-yl}guanidine;
1-[4-(3-{4-[(4-Bromophenyl)(methyl)amino]butoxy}phenyl)-1,3-thiazol-2-yl]guanidine;
1-{4-[2-(4-Chloro-2-methylphenoxy)phenyl]-1,3-thiazol-2-yl}guanidine;
1-{4-[2-(2,4-Dimethylphenoxy)phenyl]-1,3-thiazol-2-yl}guanidine;
1-{4-[2-(4-Cyclohexylphenoxy)phenyl]-1,3-thiazol-2-yl}guanidine;
1-[4-(3,5-di-tert-Butyl-4-hydroxyphenyl)-1,3-thiazol-2-yl]guanidine;
1-(4-Biphenyl-3-yl-1,3-thiazol-2-yl)guanidine;
1-[4-(4-Phenoxyphenyl)-1,3-thiazol-2-yl]guanidine;
1-[4-(4-Benzylphenyl)-1,3-thiazol-2-yl]guanidine;
1-[4-(4-Cyclohexylphenyl)-1,3-thiazol-2-yl]guanidine;
1-[4-(3-Chloro-2-hydroxy-4,6-dimethoxyphenyl)-1,3-thiazol-2-yl]guanidine;
1-[4-(2-Naphthyl)-1,3-thiazol-2-yl]guanidine;
1-(4-Biphenyl-2-yl-1,3-thiazol-2-yl)guanidine;
4-(2-{[Amino(imino)methyl]amino}-5-methyl-1,3-thiazol-4-yl)phenyl pivalate;
1-[4-(4-Hydroxyphenyl)-5-methyl-1,3-thiazol-2-yl]guanidine;
1-(5-Methyl-4-phenyl-1,3-thiazol-2-yl)guanidine;
1-(5-Ethyl-4-phenyl-1,3-thiazol-2-yl)guanidine;
1-{4-[3-(Trifluoromethyl)phenyl]-1,3-thiazol-2-yl}guanidine;
1-[4-(2,3,4-Trihydroxyphenyl)-1,3-thiazol-2-yl]guanidine;
1-[4-(2,5-Dihydroxyphenyl)-1,3-thiazol-2-yl]guanidine;
4-(2-{[Amino(imino)methyl]amino}-1,3-thiazol-4-yl)-1,2-phenylene diacetate;
N-[4-(2-{[Amino(imino)methyl]amino}-1,3-thiazol-4-yl)benzyl]acetamide;
N-[4-(2-{[Amino(imino)methyl]amino}-1,3-thiazol-4-yl)-2-methylbenzyl]acetamide;
N-[4-(2-{[Amino(imino)methyl]amino}-1,3-thiazol-4-yl)-2-chlorobenzyl]acetamide;
N-[5-(2-{[Amino(imino)methyl]amino}-1,3-thiazol-4-yl)-2-methoxybenzyl]acetamide;
1-{4-[3-(Aminomethyl)-4-methoxyphenyl]-1,3-thiazol-2-yl}guanidine;
1-[4-(3-Cyanophenyl)-1,3-thiazol-2-yl]guanidine;
1-(4-{3-[5-(Trifluoromethyl)-1,2,4-oxadiazol-3-yl]phenyl}-1,3-thiazol-2-yl)guanidine;
1-{4-[3-(Methylamino)phenyl]-1,3-thiazol-2-yl}guanidine;
1-(4-{3-[(3-Amino-1,2,4-oxadiazol-5-yl)amino]phenyl}-1,3-thiazol-2-yl)guanidine;
1-[4-(3-{[3-(Methylamino)-1,2,4-oxadiazol-5-yl]amino}phenyl)-1,3-thiazol-2-yl]guanidine;
1-(4-{4-[1-(2-Morpholin-4-ylethyl)-1H-benzimidazol-2-yl]phenyl}-1,3-thiazol-2-yl)guanidine;
1-(4-{4-[1-(1-Ethylpiperidin-3-yl)-1H-benzimidazol-2-yl]phenyl}-1,3-thiazol-2-yl)guanidine;
1-(4-{4-[1-(3-Morpholin-4-ylpropyl)-1H-benzimidazol-2-yl]phenyl}-1,3-thiazol-2-yl)guanidine;
1-[5-Methyl-4-(3-nitrophenyl)-1,3-thiazol-2-yl]guanidine;
1-[4-(2-Aminophenyl)-1,3-thiazol-2-yl]guanidine;
1-[4-(4-Cyanophenyl)-1,3-thiazol-2-yl]guanidine;
1-{4-[2-(2-Pyrrolidin-1-ylethoxy)phenyl]-1,3-thiazol-2-yl}guanidine;
1-{5-Methyl-4-[4-(2-pyrrolidin-1-ylethoxy)phenyl]-1,3-thiazol-2-yl}guanidine;
1-{4-[3-Amino-4-(benzyloxy)phenyl]-1,3-thiazol-2-yl}guanidine;
1-(4-{3-[2-(Dimethylamino)ethoxy]phenyl}-1,3-thiazol-2-yl)guanidine;
1-{5-Methyl-4-[2-(2-pyrrolidin-1-ylethoxy)phenyl]-1,3-thiazol-2-yl}guanidine;
1-{4-[3-(3-Cyanopropoxy)phenyl]-1,3-thiazol-2-yl}guanidine;
1-[5-Ethyl-4-(3-nitrophenyl)-1,3-thiazol-2-yl]guanidine;
3-(2-{[Amino(imino)methyl]amino}-1,3-thiazol-4-yl)benzenesulfonamide;
1-[4-(3,4,5-Trimethoxyphenyl)-1,3-thiazol-2-yl]guanidine;
1-[4-(3-Hydroxyphenyl)-5-methyl-1,3-thiazol-2-yl]guanidine;
3-(2-{[Amino(imino)methyl]amino}-5-methyl-1,3-thiazol-4-yl)phenyl acetate;
1-{4-[4-(Benzyloxy)-3-nitrophenyl]-1,3-thiazol-2-yl}guanidine;
1-[4-(2-Nitrophenyl)-1,3-thiazol-2-yl]guanidine;
1-[4-(2-Hydroxyphenyl)-5-methyl-1,3-thiazol-2-yl]guanidine;
4-(2-{[Amino(imino)methyl]amino}-1,3-thiazol-4-yl)phenyl acetate;
Methyl 5-(2-{[amino(imino)methyl]amino}-1,3-thiazol-4-yl)-2-hydroxybenzoate;
1-[4-(3-Aminophenyl)-5-methyl-1,3-thiazol-2-yl]guanidine;
1-[4-(3-Aminophenyl)-5-ethyl-1,3-thiazol-2-yl]guanidine;
1-(4-{3-[3-(Dimethylamino)propoxy]phenyl}-1,3-thiazol-2-yl)guanidine;
1-{4-[4-(2-Pyrrolidin-1-ylethoxy)phenyl]-1,3-thiazol-2-yl}guanidine;
5-(2-{[Amino(imino)methyl]amino}-1,3-thiazol-4-yl)-2-hydroxy-N-methylbenzamide;
3-(2-{[Amino(imino)methyl]amino}-1,3-thiazol-4-yl)benzamide;
1-{4-[4-(Cyanomethyl)phenyl]-1,3-thiazol-2-yl}guanidine;
1-(4-{4-[3-(Trifluoromethyl)-1,2,4-oxadiazol-5-yl]phenyl}-1,3-thiazol-2-yl)guanidine;
Methyl 4-(2-{[amino(imino)methyl]amino}-1,3-thiazol-4-yl)benzoate;
1-[4-(4-Methyl-3-nitrophenyl)-1,3-thiazol-2-yl]guanidine;
1-[4-(4-Chloro-3-nitrophenyl)-1,3-thiazol-2-yl]guanidine;
1-[4-(4-Methoxy-3-nitrophenyl)-1,3-thiazol-2-yl]guanidine;
1-[4-(3-Amino-4-methoxyphenyl)-1,3-thiazol-2-yl]guanidine;
1-[4-(3-Amino-4-chlorophenyl)-1,3-thiazol-2-yl]guanidine;
1-[4-(3-Amino-4-methylphenyl)-1,3-thiazol-2-yl]guanidine;
3-(2-{[Amino(imino)methyl]amino}-1,3-thiazol-4-yl)-N-methylbenzamide;
1-{4-[3-(1H-Imidazol-2-ylamino)phenyl]-1,3-thiazol-2-yl}guanidine;
1-(4-{3-[(Methylamino)methyl]phenyl}-1,3-thiazol-2-yl)guanidine;
1-[4-(3-Amino-4-fluorophenyl)-1,3-thiazol-2-yl]guanidine;
1-[4-(2-Amino-5-bromophenyl)-1,3-thiazol-2-yl]guanidine;
1-[4-(2-Methyl-5-nitrophenyl)-1,3-thiazol-2-yl]guanidine;
1-[4-(3-Fluoro-4-nitrophenyl)-1,3-thiazol-2-yl]guanidine;
1-[4-(2-Bromo-5-nitrophenyl)-1,3-thiazol-2-yl]guanidine;
N-[3-(2-{[Amino(imino)methyl]amino}-1,3-thiazol-4-yl)phenyl]-2,2,2-trifluoroacetamide;
1-[4-(3-Formamidophenyl)-1,3-thiazol-2-yl]guanidine;
1-{4-[3-(4,5-Dihydro-1H-imidazol-2-ylamino)phenyl]-1,3-thiazol-2-yl}guanidine;
1-[4-(2-Amino-5-methylphenyl)-1,3-thiazol-2-yl]guanidine;
1-[4-(4-Bromophenyl)-1,3-thiazol-2-yl]guanidine;
1-[4-(4-Bromophenyl)-5-methyl-1,3-thiazol-2-yl]guanidine;
1-{4-[4-(Trifluoromethyl)phenyl]-1,3-thiazol-2-yl}guanidine;
1-[4-(2-Methyl-1H-indol-5-yl)-1,3-thiazol-2-yl]guanidine;
1-[4-(4-Chlorophenyl)-5-methyl-1,3-thiazol-2-yl]guanidine;
1-[4-(3-Aminophenyl)-5-butyl-1,3-thiazol-2-yl]guanidine;
1-[4-(3-Aminophenyl)-5-pentyl-1,3-thiazol-2-yl]guanidine;
4-(2-{[Amino(imino)methyl]amino}-5-methyl-1,3-thiazol-4-yl)phenyl acetate;
1-[4-(2,4,5-Trimethylphenyl)-1,3-thiazol-2-yl]guanidine;
1-{4-[3-(Cyanoamino)phenyl]-1,3-thiazol-2-yl}guanidine;
1-[4-(2-oxo-2,3-dihydro-1H-indol-5-yl)-1,3-thiazol-2-yl]guanidine;
1-[5-Methyl-4-(2-oxo-2,3-dihydro-1H-indol-5-yl)-1,3-thiazol-2-yl]guanidine;
1-[4-(2,5-Dichlorophenyl)-1,3-thiazol-2-yl]guanidine;
1-[4-(4-Acetyl-6-chloro-3,4-dihydro-2H-1,4-benzoxazin-8-yl)-1,3-thiazol-2-yl]guanidine;
1-{4-[6-Chloro-3-oxo-4-(3-phenoxypropyl)-3,4-dihydro-2H-1,4-benzoxazin-8-yl]-1,3-thiazol-2-yl}guanidine;
1-[4-(1-Acetyl-2,3-dihydro-1H-indol-5-yl)-1,3-thiazol-2-yl]guanidine;
1-{4-[6-Chloro-4-(2-morpholin-4-ylethyl)-3-oxo-3,4-dihydro-2H-1,4-benzoxazin-8-yl]-1,3-thiazol-2-yl}guanidine;
1-[4-(2,4-Dichlorophenyl)-1,3-thiazol-2-yl]guanidine;
1-{4-[3-(1,3-Dioxo-1,3-dihydro-2H-isoindol-2-yl)phenyl]-1,3-thiazol-2-yl}guanidine;
1-[4-(1-Acetyl-2-methyl-2,3-dihydro-1H-indol-5-yl)-1,3-thiazol-2-yl]guanidine;
1-[4-(1,3-Benzodioxol-5-yl)-1,3-thiazol-2-yl]guanidine;
1-[5-Methyl-4-(1-propionyl-2,3-dihydro-1H-indol-5-yl)-1,3-thiazol-2-yl]guanidine;
1-[4-(2,4-Dimethylphenyl)-1,3-thiazol-2-yl]guanidine;
1-[4-(1-Isobutyryl-2,3-dihydro-1H-indol-5-yl)-1,3-thiazol-2-yl]guanidine;
1-[4-(1-Isobutyryl-2,3-dihydro-1H-indol-5-yl)-5-methyl-1,3-thiazol-2-yl]guanidine;
1-(4-{3-[(1,3-Dioxo-1,3-dihydro-2H-isoindol-2-yl)methyl]phenyl}-1,3-thiazol-2-yl)guanidine;
1-{4-[1-(Cyclopropylcarbonyl)-2,3-dihydro-1H-indol-5-yl]-1,3-thiazol-2-yl}guanidine;
1-{4-[1-(Cyclopropylcarbonyl)-2-methyl-2,3-dihydro-1H-indol-5-yl]-1,3-thiazol-2-yl}guanidine;
1-[4-(6-Chloro-4-methyl-3-oxo-3,4-dihydro-2H-1,4-benzoxazin-8-yl)-1,3-thiazol-2-yl]guanidine;
1-{4-[1-(Cyclohexylcarbonyl)-2,3-dihydro-1H-indol-5-yl]-5-methyl-1,3-thiazol-2-yl}guanidine;
1-{4-[1-(Methylsulfonyl)-2,3-dihydro-1H-indol-5-yl]-1,3-thiazol-2-yl}guanidine;
1-{4-[2-Methyl-1-(methylsulfonyl)-2,3-dihydro-1H-indol-5-yl]-1,3-thiazol-2-yl}guanidine;
1-[4-(1-Acetyl-2,3-dihydro-1H-indol-5-yl)-5-methyl-1,3-thiazol-2-yl]guanidine;
1-{5-Methyl-4-[1-(methylsulfonyl)-2,3-dihydro-1H-indol-5-yl]-1,3-thiazol-2-yl}guanidine;
1-[4-(1-Propionyl-2,3-dihydro-1H-indol-5-yl)-1,3-thiazol-2-yl]guanidine;
1-[4-(2-Methyl-1-propionyl-2,3-dihydro-1H-indol-5-yl)-1,3-thiazol-2-yl]guanidine;
1-[4-(3-Oxo-3,4-dihydro-2H-1,4-benzoxazin-6-yl)-1,3-thiazol-2-yl]guanidine;
1-[4-(2-Methyl-1H-imidazol-4-yl)-1,3-thiazol-2-yl]guanidine;
N-{[5-(2-{[Amino(imino)methyl]amino}-1,3-thiazol-4-yl)-2-furyl]methyl}acetamide;
1-(4-{5-[(Cyanoamino)methyl]-2-furyl}-1,3-thiazol-2-yl)guanidine;
N-{[5-(2-{[Amino(imino)methyl]amino}-1,3-thiazol-4-yl)-2-furyl]methyl}propanamide;
N-{[5-(2-{[Amino(imino)methyl]amino}-1,3-thiazol-4-yl)-2-furyl]methyl}butanamide;
N-{[5-(2-{[Amino(imino)methyl]amino}-1,3-thiazol-4-yl)-2-furyl]methyl}-2-methylpropanamide;
N-{[5-(2-{[Amino(imino)methyl]amino}-1,3-thiazol-4-yl)-2-furyl]methyl}pentanamide;
1-{4-[5-(Aminomethyl)-2-furyl]-1,3-thiazol-2-yl}guanidine;
N-{[5-(2-{[Amino(imino)methyl]amino}-1,3-thiazol-4-yl)-2-thienyl]methyl}acetamide;
N-[(2′-{[Amino (imino)methyl]amino}-4,4′-bi-1,3-thiazol-2-yl)methyl]acetamide;
N-[(2-{[Amino(imino)methyl]amino}-4,5′-bi-1,3-thiazol-2′-yl)methyl]acetamide;
N-{[5-(2-{[Amino(imino)methyl]amino}-1,3-thiazol-4-yl)-1,3,4-oxadiazol-2-yl]methyl}acetamide;
N-{[5-(2-{[Amino(imino)methyl]amino}-1,3-thiazol-4-yl)-4H-1,2,4-triazol-3-yl]methyl}acetamide;
1-{4-[5-(2-Methyl-1H-imidazol-5-yl)-2-furyl]-1,3-thiazol-2-yl}guanidine;
1-{4-[5-(5-Amino-4H-1,2,4-triazol-3-yl)-2-furyl]-1,3-thiazol-2-yl}guanidine;
Methyl 5-(2-{[amino(imino)methyl]amino}-1,3-thiazol-4-yl)-2-furoate;
Methyl[5-(2-{[amino(imino)methyl]amino}-1,3-thiazol-4-yl)-2-furyl]acetate;
1-(4-{5-[(1,3-Dioxo-1,3-dihydro-2H-isoindol-2-yl)methyl]-4H-1,2,4-triazol-3-yl}-1,3-thiazol-2-yl)guanidine;
1-{4-[(2,5-Dioxopyrrolidin-1-yl)carbonyl]-1,3-thiazol-2-yl}guanidine;
3-{[(2-{[Amino(imino)methyl]amino}-4-methyl-1,3-thiazol-5-yl)carbonyl]amino}benzoic acid;
Ethyl 3-{[(2-{[amino(imino)methyl]amino}-4-methyl-1,3-thiazol-5-yl)carbonyl]amino}benzoate;
as a free base or a pharmaceutically acceptable salt, solvate or solvate of a salt thereof.

2. A compound according to claim 1, wherein R² is selected from hydrogen and C₁₋₆ alkyl.

3. A compound according to claim 1, wherein R² is selected from hydrogen, methyl and ethyl.

4. A compound according to claim 1, wherein Q is selected from phenyl, thiazole and C₀₋₃alkylCONR⁴R⁵.

5. A compound according to claim 4, wherein Q is thiazole.

6. A compound according to claim 5, wherein R² is selected from hydrogen and methyl.

7. A compound according to claim 5, wherein m is 1 or 2.

8. A compound according to claim 5, wherein R³ is independently selected from NR⁴R⁵, C₁₋₆alkyl and heteroaryl, wherein said heteroaryl may be optionally substituted with one or more A.

9. A compound according to claim 8, wherein R³ is NR⁴R⁵, wherein said R⁴ and R⁵ represents hydrogen.

10. A compound according to claim 8, wherein R³ is heteroaryl, substituted with one or more A.

11. A compound according to claim 10, wherein R³ is pyridine, substituted with two halogens.

12. A compound according to claim 10, wherein A is independently selected from NR⁶R⁷ and hydrogen.

13. A compound according to claim 12, wherein R⁶ and R⁷ is hydrogen.

14. A compound according to claim 8, wherein R³ is NR⁴R⁵, wherein R⁴ represents hydrogen and R⁵ represents aryl, said aryl optionally substituted with one or more A.

15. A compound according to claim 14, wherein R⁵ is aryl, substituted with one or more A, said A being selected from halogen, nitro, CN, OR⁶, NR⁶R⁷, COR⁶ and CO₂R⁶.

16. A compound according to claim 15, wherein R⁶ and R⁷ are independently selected from hydrogen and methyl.

17. A compound according to claim 15, wherein R⁶ is C₀₋₆alkylaryl.

18. A compound according to claim 8, wherein R³ is NR⁴R⁵, wherein R⁴ and R⁵ represents methyl.

19. A compound according to claim 8, wherein R³ is NR⁴R⁵, wherein R⁴ and R⁵ independently is selected from hydrogen and C₂₋₆alkenyl.

20. A compound according to claim 8, wherein R³ is NR⁴R⁵, wherein R⁴ and R⁵ independently is selected from hydrogen and C₀₋₆alkylheteroaryl.

21. A compound according to claim 20, wherein said C₀₋₆alkylheteroaryl is pyridine.

22. A compound according to claim 1, wherein Q is phenyl.

23. A compound according to claim 22, wherein m is 1.

24. A compound according to claim 22, wherein R³ is selected from nitro, NR⁴R⁵, NR⁴COR⁵, CONR⁴R⁵ and NR⁴(SO₂)R⁵.

25. A compound according to claim 24, wherein R³ is NR⁴R⁵, wherein said R⁴ and R⁵ are independently selected from C₀₋₆alkylaryl and C₀₋₆alkylC₃₋₆heterocyclyl.

26. A compound according to claim 24, wherein R³ is NR⁴R⁵, wherein said R⁴ and R⁵ are independently selected from hydrogen and C₀₋₆alkylaryl, said C₀₋₆alkylaryl optionally substituted with one or more A.

27. A compound according to claim 26, wherein said A is independently selected from OR⁶, CO₂R⁶ and halogen.

28. A compound according to claim 27, wherein said R⁶ is selected from hydrogen and C₀₋₆alkylaryl.

29. A compound according to claim 24, wherein R³ is NR⁴R⁵, wherein said R⁴ and R⁵ together form a 5 membered heterocyclic ring containing one N heteroatom.

30. A compound according to claim 24, wherein R³ is NR⁴R⁵, wherein said R⁴ represents hydrogen and said R⁵ represents C₂₋₆alkenyl optionally substituted with one A.

31. A compound according to claim 30, wherein said A represents C₀₋₆alkylaryl, substituted with NR⁶NR⁷, said R⁶ and R⁷ being C₁₋₆alkyl.

32. A compound according to claim 24, wherein R³ is NR⁴(SO₂)R⁵.

33. A compound according to claim 32, wherein R⁴ represents hydrogen and R⁵ represents C₀₋₆alkylaryl, said C₀₋₆alkylaryl being substituted with one or more A.

34. A compound according to claim 33, wherein A represents C₀₋₆alkylaryl, substituted with one OR⁶ and R⁶ represents methyl.

35. A compound according to claim 24, wherein R³ is NR⁴COR⁵.

36. A compound according to claim 35, wherein R⁴ represents hydrogen and R⁵ represents C₀₋₆alkylaryl or C₀₋₆alkylheteroaryl, said C₀₋₆alkylaryl or C₀₋₆alkylheteroaryl being substituted with one or more A.

37. A compound according to claim 36, wherein A is independently selected from halogen, OR⁶, C₁₋₆alkyl, C₀₋₆alkylaryl, heteroaryl, said C₁₋₆alkyl, C₀₋₆alkylaryl and heteroaryl being optionally substituted with NR⁶R⁷.

38. A compound according to claim 37, wherein R⁶ and R⁷ is independently selected from methyl and C₀₋₆alkylaryl.

39. A compound according to claim 24, wherein R³ is CONR⁴R⁵.

40. A compound according to claim 39, wherein R⁴ and R⁵ is independently selected from hydrogen and C₀₋₆alkylC₃₋₆heterocyclyl.

41. A compound according to claim 39, wherein R⁴ and R⁵ together form a 6 membered heterocyclic ring containing one or more N heteroatoms, which heterocyclic ring is substituted by one or more A.

42. A compound according to claim 41, wherein said A is C₀₋₆alkylaryl optionally substituted with OR⁶.

43. A compound according to claim 42, wherein R⁶ is hydrogen.

44. A compound according to claim 4, wherein Q is C₀₋₃alkylCONR⁴R⁵, and m is 0.

45. A compound according to claim 44, wherein R⁴ is hydrogen and R⁵ is C₀₋₆alkylaryl.

46. A compound according to claim 44, wherein R⁴ is hydrogen and R⁵ is C₀₋₆alkylheteroaryl, optionally substituted with C₁₋₆alkyl.

47. A compound selected from:
2-{[Amino(imino)methyl]amino}-N-1-anthryl-1,3-thiazole-4-carboxamide;
2-{[Amino(imino)methyl]amino}-N-(9-ethyl-9H-carbazol-3-yl)-1,3-thiazole-4-carboxamide;
N-[3-(2-{[Amino(imino)methyl]amino}-4-methyl-1,3-thiazol-5-yl)phenyl]-4-(benzyloxy)benzamide;
N-[3-(2-{[Amino(imino)methyl]amino}-4-methyl-1,3-thiazol-5-yl)phenyl]-4′-methoxybiphenyl-4-sulfonamide;
N-[3-(2-{[Amino(imino)methyl]amino}-4-methyl-1,3-thiazol-5-yl)phenyl]-4-(1,2,3-thiadiazol-4-yl)benzamide;
N-[3-(2-{[Amino(imino)methyl]amino}-4-methyl-1,3-thiazol-5-yl)phenyl]-3-chloro-6-ethyl-1-benzothiophene-2-carboxamide;
N-[3-(2-{[Amino(imino)methyl]amino}-4-methyl-1,3-thiazol-5-yl)phenyl]-2-naphthamide;
Benzyl 4-({[4-(2-{[amino(imino)methyl]amino}-4-ethyl-1,3-thiazol-5-yl)phenyl]amino}methyl)piperidine-1-carboxylate;
N″-{5-[4-(Dibenzylamino)phenyl]-4-ethyl-1,3-thiazol-2-yl}guanidine;
N-(5-{3-[(2-Bromo-6-hydroxybenzyl)amino]phenyl}-4-methyl-1,3-thiazol-2-yl)guanidine;
N-[5-(4-{[4-(2-Hydroxyphenyl)piperazin-1-yl]carbonyl}phenyl)-4-methyl-1,3-thiazol-2-yl]guanidine;
N-{2′-[(3,4-dichlorophenyl)amino]-4-methyl-5,5′-bi-1,3-thiazol-2-yl}guanidine;
N-{4-methyl-2′-[(4-nitrophenyl)amino]-5,5′-bi-1,3-thiazol-2-yl}guanidine;
N-{2′-[(4-cyanophenyl)amino]-4-methyl-5,5′-bi-1,3-thiazol-2-yl}guanidine;
N-{2′-[(4-hydroxyphenyl)amino]-4-methyl-5,5′-bi-1,3-thiazol-2-yl}guanidine;
4-[(2′-{[amino(imino)methyl]amino}-4′-methyl-5,5′-bi-1,3-thiazol-2-yl)amino]benzoic acid;
N-{2′-[(4-acetylphenyl)amino]-4-methyl-5,5′-bi-1,3-thiazol-2-yl}guanidine;
N-{2′-[(2,4-dimethoxyphenyl)amino]-4-methyl-5,5′-bi-1,3-thiazol-2-yl}guanidine;
4-(2-{[Amino(imino)methyl]amino}-4-methyl-1,3-thiazol-5-yl)-N-(4-pyrrolidin-1-ylbutyl)benzamide;
Benzyl 4-{[(4-{2-[(diaminomethylene)amino]-4-methyl-1,3-thiazol-5-yl}phenyl)amino]methyl}piperidine-1-carboxylate;
2-(5-{4-[Benzyl(piperidin-4-ylmethyl)amino]phenyl}-4-methyl-1,3-thiazol-2-yl)guanidine;
2-{5-[4-({(2E)-3-[4-(Dimethylamino)phenyl]prop-2-en-1-yl}amino)phenyl]-4-methyl-1,3-thiazol-2-yl}guanidine;
1-[5-(2-Nitrophenyl)-1,3-thiazol-2-yl]guanidine;
1-[4-(4-Pyrrolidin-1-ylphenyl)-1,3-thiazol-2-yl]guanidine;
1-{2-[(4-Aminophenyl)amino]-4′-methyl-4,5′-bi-1,3-thiazol-2′-yl}guanidine;
1-{4′-Methyl-2-[(4-phenoxyphenyl)amino]-4,5′-bi-1,3-thiazol-2′-yl}guanidine;
1-{2-[(4-Methoxyphenyl)amino]-4′-methyl-4,5′-bi-1,3-thiazol-2′-yl}guanidine;
1-{4′-Methyl-2-[(4-nitrophenyl)amino]-4,5′-bi-1,3-thiazol-2′-yl}guanidine;
1-[4′-Methyl-2-(pyridin-4-ylamino)-4,5′-bi-1,3-thiazol-2′-yl]guanidine;
1-[2-(Dimethylamino)-4′-methyl-4,5′-bi-1,3-thiazol-2′-yl]guanidine;
1-[2-(Allylamino)-4′-methyl-4,5′-bi-1,3-thiazol-2′-yl]guanidine;
1-[2-(2,6-Dichloropyridin-4-yl)-4′-methyl-4,5′-bi-1,3-thiazol-2′-yl]guanidine;
N-[4-(2-{[Amino(imino)methyl]amino}-4-methyl-1,3-thiazol-5-yl)phenyl]-5-fluoro-1H-indole-2-carboxamide;
N-[4-(2-{[amino(imino)methyl]amino}-4-methyl-1,3-thiazol-5-yl)phenyl]-4-{[methyl(phenyl)amino]methyl}benzamide;
2-{[Amino(imino)methyl]amino}-N-(9-oxo-9H-fluoren-2-yl)-1,3-thiazole-4-carboxamide;
2-{[Amino(imino)methyl]amino}-N-1H-indol-5-yl-1,3-thiazole-4-carboxamide;
1-(2′-Amino-4′-methyl-4,5′-bi-1,3-thiazol-2-yl)guanidine;
1-[2-(3,5-Diamino-6-chloropyrazin-2-yl)-4′-methyl-4,5′-bi-1,3-thiazol-2′-yl]guanidine;
as a free base or a pharmaceutically acceptable salt, solvate or solvate of a salt thereof.

48. A pharmaceutical formulation comprising as active ingredient a therapeutically effective amount of a compound according to claim 1 in association with a pharmaceutically acceptable excipient, carrier or diluent.

49-99. (canceled)

100. A method of inhibiting activity of BACE comprising contacting said BACE with a compound of claim 1.

101. A method of treating or preventing an Aβ-related pathology in a mammal, comprising administering to said patient a therapeutically effective amount of a compound of formula I: 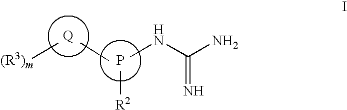
wherein
P is thiazole;
Q is independently selected from phenyl, thiazole, C₀₋₃alkylCONR⁴R⁵, C₀₋₃alkylNR⁴COR⁵, C₀₋₃alkylNR⁴(SO₂)R⁵, and C₀₋₃alkyl(SO₂)NR⁴R⁵;
R² is independently selected from hydrogen, halogen, C₁₋₆alkyl, CN, C₀₋₆alkylOR⁴, fluoromethyl, difluoromethyl, trifluoromethyl, fluoromethoxy, difluoromethoxy, trifluoromethoxy, C₂₋₆alkenyl and C₂₋₆alkynyl;
R³ is independently selected from halogen, nitro, CHO, CN, OC₁₋₆alkylCN, OR⁴, OC₁₋₆alkylOR⁴, fluoromethyl, difluoromethyl, trifluoromethyl, fluoromethoxy, difluoromethoxy, trifluoromethoxy, NR⁴R⁵, OC₁₋₆alkylNR⁴R⁵, NR⁴COR⁵, CO₂R⁴, CONR⁴R⁵, OC₁₋₆alkylCONR⁴R⁵, OC₁₋₆alkylNR⁴(CO)R⁵, NR⁴(CO)R⁵, O(CO)NR⁴R⁵, NR⁴(CO)OR⁵, NR⁴(CO)NR⁴R⁵, O(CO)R⁴, COR⁴, OC₁₋₆alkylCOR⁴, SR⁴, (SO₂)NR⁴R⁵, OC₁₋₆alkylNR⁴(SO₂)R⁵, OC₀₋₆alkyl(SO₂)NR⁴R⁵, (SO)NR⁴R⁵, OC₁₋₆alkyl(SO)NR⁴R⁵, SO₃R⁴, NR⁴(SO)R⁵, NR⁴(SO₂)R⁵, OC₁₋₆alkylNR⁴(SO)R⁵, OC₀₋₆alkylSO₂R⁴, SO₂R⁴, SOR⁴, C₁₋₆alkyl, C₂₋₆alkenyl, C₂₋₆alkynyl, C₃₋₆cycloalkyl, aryl and heteroaryl, wherein said C₁₋₆alkyl, C₂₋₆alkenyl, C₂₋₆alkynyl, C₃₋₆cycloalkyl, aryl or heteroaryl may be optionally substituted with one or more A;
R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylC₃₋₆heterocyclyl, C₀₋₆alkylaryl, C₀₋₆alkylheteroaryl and C₁₋₆alkylNR⁶R⁷, wherein said C₁₋₆alkyl, C₂₋₆alkenyl, C₀₋₆alkylC₃₋₆heterocyclyl, C₀₋₆alkylaryl or C₀₋₆alkylheteroaryl may be optionally substituted by one or more A; or
R⁴ and R⁵ may together form a 5 or 6 membered heterocyclic ring containing one or more heteroatoms selected from N, O or S, which heterocyclic ring may be optionally substituted by one or more A;
A is independently selected from oxo, halogen, nitro, CN, OR⁶, C₁₋₆alkyl, C₀₋₆alkylaryl, heteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, NR⁶R⁷, CONR⁶R⁷, NR⁶(CO)R⁷, O(CO)R⁶, CO₂R⁶, COR⁶, (SO₂)NR⁶R⁷, NR⁶SO₂R⁷, SO₂R⁶, SOR⁶, OSO₂R⁶ and SO₃R⁶, wherein said C₁₋₆alkyl, C₀₋₆alkylaryl, heteroaryl, C₀₋₆alkylheterocyclyl or C₀₋₆alkylC₃₋₆cycloalkyl may be optionally substituted with one or more substituents independently selected from halogen, nitro, cyano, OR⁶, C₁₋₆alkyl, fluoromethyl, difluoromethyl, trifluoromethyl, fluoromethoxy, difluoromethoxy, trifluoromethoxy, and NR⁶R⁷;
R⁶ and R⁷ are independently selected from hydrogen, C₁₋₆alkyl, C₀₋₆alkylaryl, fluoromethyl, difluoromethyl and trifluoromethyl, or
R⁶ and R⁷ may together form an optionally substituted 5, 6 or 7 membered heterocyclic ring containing one or more heteroatoms selected from N, O or S;
m is 0, 1, 2 or 3;
provided that when Q is C₀₋₃alkylCONR⁴R⁵ and R⁴ or R⁵ is C₀₋₆alkylC₃₋₆heterocyclyl, said C₀₋₆alkylC₃₋₆heterocyclyl is not benzotriazole; and
provided that when Q is C₀₋₃alkylCONR⁴R⁵, C₀₋₃alkylNR⁴COR⁵, C₀₋₃alkylNR⁴(SO₂)R⁵ or C₀₋₃alkyl(SO₂)NR⁴R⁵, m is 0.

102. The method of claim 101, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

103. The method of claim 101, wherein said mammal is a human.

104-149. (canceled)
